(19) United States
(12) Patent Application Publication
(10) Pub. No.: US 2004/0254599 A1
(43) Pub. Date: Dec. 16, 2004

(54) Method and apparatus for pre-lancing stimulation of puncture site

(76) Inventors: Michael V. Lipoma, Villa Rica, GA (US); Avi M. Robbins, Longwood, FL (US); Carl E. Griffin, Marietta, GA (US); David R. Buenger, Roswell, GA (US)

Correspondence Address: GARDNER GROFF, P.C., PAPER MILL VILLAGE, BUILDING 23, 600 VILLAGE TRACE, SUITE 300, MARIETTA, GA, 30067, US

(21) Appl. No.: 10/809,194

(22) Filed: Mar. 25, 2004

Related U.S. Application Data
(60) Provisional application No. 60/457,518, filed on Mar. 25, 2003.

(51) Int. Cl. A61B 17/14

(52) U.S. Cl. 606/181

(57) ABSTRACT

A method of reducing perceived pain resulting from puncturing of skin at a puncture site. A sensory distraction is generated at or adjacent the puncture site, and the skin is punctured at the puncture site simultaneously with or after the generation of the sensory distraction. Also, a device for penetrating the skin of a human or animal subject, including means for penetrating the skin at a puncture site, and means for generating a sensory distraction at or adjacent the puncture site. Furthermore, an endcap with penetration depth adjustability is provide that includes keepers for receiving new lancets tip-first for safety when reloading multi-use lancing devices.

### CROSS-REFERENCE TO RELATED APPLICATION

This application claims the priority benefit of U.S. Provisional Application Ser. No. 60/457,518, filed Mar. 25, 2003, which is hereby incorporated by reference herein.

### FIELD OF THE INVENTION

The present invention relates generally to medical devices and procedures, and more particularly to methods and devices for reducing the sensation of pain resulting from punctures of the skin.

### BACKGROUND OF THE INVENTION

Many medical procedures require puncturing of the skin, and sometimes underlying tissues, of an animal or human subject. For example, when administering a shot or drawing blood for testing using a hypodermic syringe, the needle of the syringe typically must puncture the subject's skin and enter underlying subcutaneous tissues. Needles are also used to collect blood from blood donors. Similarly, a sharp lancet tip is commonly used to puncture the subject's skin for sampling of blood or other body fluid, as for example in blood glucose monitoring by diabetics.

Many subjects find these procedures to be painful and, as a result, avoid undertaking them even when the procedure is medically advisable. For example, many diabetics do not sample their blood sugar as often as is recommended, potentially leading to adverse health effects. Likewise, some persons avoid getting shots, such as flu shots, because they fear the pain of the needle-stick, potentially resulting in their contracting the flu or other ailment that could have been prevented had they taken the shot.

Thus, it has been found that a need exists for a method and apparatus for reducing the sensation of pain from skin-puncturing procedures. It is to a method and apparatus meeting this and other needs that the present invention is primarily directed.

### SUMMARY OF THE INVENTION

The present invention provides a method and apparatus for reducing the sensation of pain from skin-puncturing procedures. Generally described, the method and apparatus of the present invention confuse or distract the nerves at or adjacent the puncture site prior to or simultaneously with the puncturing procedure. For example, an impact, vibration, acoustical signal or electrical stimulation may be imparted at or adjacent the puncture site prior to or simultaneously with the puncturing procedure. It has been found that nerve distraction or confusion generated in this manner lessens the sensation of pain resulting from the puncturing procedure in many subjects.

In one aspect, the present invention is a method of reducing perceived pain resulting from puncturing of skin at a puncture site. The method preferably includes generating a sensory distraction at or adjacent the puncture site; and puncturing the skin at the puncture site simultaneously with or after the generation of the sensory distraction. In example embodiments, the step of generating a sensory distraction comprises impacting a first element such as a stop member with a second element such as a lancet drive member to generate a vibration for transmission to the puncture site. In other example embodiments, the step of generating a sensory distraction comprises impacting the puncture site with a stimulator member, for example, by implementing a varied length scheme or a two-stage scheme for timing the puncturing simultaneously with or after the stimulator impact. And in still other example embodiments, the step of generating a sensory distraction comprises vibration, sound, impact, electrical stimulation, heat, light, taste, or smell. In another aspect, the present invention is a device for penetrating the skin of a human or animal subject. The device preferably includes means for penetrating the skin at a puncture site, such as a lancet or hypodermic needle; and means for generating a sensory distraction at or adjacent the puncture site. In example embodiments, the sensory distraction means comprises a first element such as a stop member for impacting a second element such as a lancet drive member and generating a vibration for transmission to the puncture site. In other example embodiments, the sensory distraction means comprises a stimulator member. For example, in a varied length scheme embodiment, the stimulator member is longer than the lancet, and a drive spring is provided for driving both the stimulator member and the lancet. As another example, in two-stage scheme embodiment, there are provided a stimulator drive spring for driving the stimulator member and a separate lancet drive spring for driving the lancet. And in still other example embodiments, the sensory distraction means comprises a transducer or contact, for example, for generating vibration, sound, impact, electrical stimulation, heat, light, taste, or smell, or a plurality of pointed teeth, for generating the sensory distraction.

And in still another aspect of the invention, the lancing device has a penetration depth adjustment mechanism comprising an endcap that rotates relative to the lancing device housing. The lancet includes at least one engagement surface, and the endcap has a plurality of stop surfaces that can be selectively aligned with and engaged by the lancet engagement surface to limit forward lancet movement at different depths. In addition, the carriage preferably has a flared proximal section with a flared bore that receives the endcap stop surfaces not aligned with and engaged by the lancet body engagement surface. And more preferably, the endcap has a plurality of keeper arms extending distally therefrom for coupling the lancet to the endcap, the keeper arms including inward retainer tabs that can pass through keeper slots formed in a ridge on the lancet and that catch on the ridge to hold the lancet to the endcap, the keeper arms further having outward guide tabs that are received in female key guide slots in the carrier.

These and other aspects, features, and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWING FIGURES

FIG. 1 is an exploded perspective view of a lancing device according to a first example embodiment of the present invention.

FIG. 2 is a cross-sectional view of the lancing device of FIG. 1, in an uncocked, rest position.

FIG. 3 is a cross-sectional view of the lancing device of FIG. 1, in a cocked or armed position.

FIG. 4 is a cross-sectional view of the lancing device of FIG. 1, in a firing or activated position.

FIG. 5 is a cross-sectional view of the lancing device of FIG. 1, in a piston/lancet impact position.

FIG. 6 is a cross-sectional view of the lancing device of FIG. 1, in a piston stop position, just before decoupling.

FIG. 7 is a cross-sectional view of the lancing device of FIG. 1, in an extended or puncturing position, with the lancet decoupled from the piston.

FIG. 8 is an exploded side view of an endcap of the lancing device of FIG. 1, showing an inner cap and an outer cap.

FIG. 9 is a side view of the endcap of FIG. 8, showing the outer cap assembled onto the inner cap in a first depth position.

FIG. 10 is a side view of the endcap of FIG. 8, showing the outer cap rotated to a second depth position.

FIG. 11 is a side view of the endcap of FIG. 8, showing the outer cap rotated to a third depth position.

FIG. 12 is a cross-sectional view of a portion of the endcap taken at line B-B of FIG. 11.

FIG. 13 is a side view of the endcap of FIG. 8, showing the outer cap rotated to a fourth depth position.

FIG. 14 is a side view of the endcap of FIG. 8, showing the outer cap rotated to a fifth depth position.

FIG. 15 is a perspective view of a lancing device according to a second example embodiment of the present invention.

FIG. 16 is a longitudinal cross-section view of the lancing device of FIG. 15, with the lancet carrier in a retracted position for cocking the device.

FIG. 17 is a longitudinal cross-section view of the lancing device of FIG. 15, in an armed state.

FIG. 18 is a longitudinal cross-section view of the lancing device of FIG. 15, with the lancet in an extended position of the lancing stroke for puncturing the skin.

FIG. 19 is an exploded perspective view of a lancing device according to a third example embodiment of the present invention.

FIG. 20 is a front perspective view of a carrier of the lancing device of FIG. 19.

FIG. 21 is a rear perspective view of a carrier of the lancing device of FIG. 19.

FIG. 22 is a front perspective view of a lancet of the lancing device of FIG. 19.

FIG. 23 is a rear perspective view of an endcap of the lancing device of FIG. 19.

FIG. 24 is a longitudinal cross-section view of the lancing device of FIG. 19, with the lancet and carrier in a rest position.

FIG. 25 is a longitudinal cross-section view of the lancing device of FIG. 19, with the lancet and carrier in a retracted position.

FIG. 26 is a cross-section view of the lancing device taken at line G-G of FIG. 25.

FIG. 27 is a cross-section detail view, Detail “H” of FIG. 26, showing the cocking arm catch being retained in place.

FIG. 28 is a longitudinal cross-section view of the lancing device of FIG. 19, with the lancet and carrier in an activated or firing position.

FIG. 29 is a cross-section view of the lancing device taken at line J-J of FIG. 28.

FIG. 30 is a cross-section detail view, Detail “K” of FIG. 29, showing the cocking arm catch being released for firing.

FIG. 31 is a longitudinal cross-section view of the lancing device of FIG. 19, with the carrier in a stopped position.

FIG. 32 is a longitudinal cross-section view of the lancing device of FIG. 19, with the carrier stopped and the lancet continuing to the extended or puncturing position.

FIG. 33 is an exploded perspective view of the carrier (in cross section), lancet, and endcap of the lancing device of FIG. 19.

FIG. 34 is a perspective view of the carrier (in cross section), lancet, and endcap of FIG. 33, with the endcap in a safety position.

FIG. 35 is a perspective view of the carrier (in cross section), lancet, and endcap of FIG. 33, with the endcap in a shallow puncturing depth position.

FIG. 36 is a perspective view of the carrier (in cross section), lancet, and endcap of FIG. 35, with the lancet fired and in the puncturing position.

FIG. 37 is a perspective view of the carrier (in cross section), lancet, and endcap of FIG. 33, with the endcap in a deep puncturing depth position.

FIG. 38 is a perspective view of the carrier (in cross section), lancet, and endcap of FIG. 37, with the lancet fired and in the puncturing position.

FIG. 39 is a front view of the carrier and lancet of FIG. 19.

FIG. 40 is a front view of an alternative lancet for use with the carrier and endcap of FIG. 33.

FIG. 41 is an exploded perspective view of a lancing device according to a fourth example embodiment of the present invention.

FIG. 42A is a rear perspective view of a carrier of the lancing device of FIG. 41.

FIG. 42B is a front perspective view of a carrier of the lancing device of FIG. 41.

FIG. 43 is a front perspective view of a lancet of the lancing device of FIG. 41.

FIG. 44 is a rear perspective view of a lancet of the lancing device of FIG. 41.

FIG. 45 is a front perspective view of an endcap of the lancing device of FIG. 41.

FIG. 46 is a rear perspective view of an endcap of the lancing device of FIG. 41.

FIG. 47 is a longitudinal cross-section view of the lancing device of FIG. 41, with the lancet and carrier in a rest position.

FIG. 48 is a longitudinal cross-section view of the lancing device of FIG. 41, with the lancet and carrier in a retracted or cocked position.

FIG. 49 is a cross-section view of the lancing device taken at line D-D of FIG. 48.

FIG. 50 is a cross-section detail view, Detail “E” of FIG. 49, showing the cocking arm catch being retained in place.

FIG. 51 is a longitudinal cross-section view of the lancing device of FIG. 41, with the lancet and carrier in an activated or firing position.

FIG. 52 is a cross-section view of the lancing device taken at line C-C of FIG. 51.

FIG. 53 is a cross-section detail view, Detail “F” of FIG. 51, showing the cocking arm catch being released for firing.

FIG. 54 is a longitudinal cross-section view of the lancing device of FIG. 41, with the carrier in a stopped, pre-stimulation position.

FIG. 55 is a longitudinal cross-section view of the lancing device of FIG. 41, with the carrier stopped and the lancet continuing to the extended or puncturing position.

FIG. 56 is an exploded perspective view of the carrier (in cross section), lancet, and endcap of the lancing device of FIG. 41.

FIG. 57 is a perspective view of the carrier (in cross section), lancet, and endcap of FIG. 56, with the lancet and endcap coupled together.

FIG. 58 is a perspective view of the carrier (in cross section), lancet, and endcap of FIG. 57, with the lancet/endcap coupled to the carrier.

FIG. 59 is a perspective view of the carrier (in cross section), lancet, and endcap of FIG. 56, with the endcap in a shallow puncturing depth position.

FIG. 60 is a perspective view of the carrier (in cross section), lancet, and endcap of FIG. 59, with the lancet fired and in the puncturing position.

FIG. 61 is a perspective view of the carrier (in cross section), lancet, and endcap of FIG. 56, with the endcap in a deep puncturing depth position.

FIG. 62 is a perspective view of the carrier (in cross section), lancet, and endcap of FIG. 61, with the lancet fired and in the puncturing position.

FIG. 63 shows a longitudinal cross-section view of a lancing device according to a fifth example embodiment of the present invention.

FIG. 64 shows a longitudinal cross-section view of a lancing device according to a sixth example embodiment of the present invention.

FIG. 65 shows a perspective view of a lancing device according to a seventh example embodiment of the present invention.

FIG. 66 shows a perspective view of a lancing device according to an eighth example embodiment of the present invention.

FIG. 67 shows a perspective view of a syringe according to a ninth example embodiment of the present invention.

### DETAILED DESCRIPTION OF EXAMPLE EMBODIMENTS

Referring now to the drawing figures, wherein like reference numerals represent like parts throughout, preferred forms of the present invention will now be described. It is to be understood that this invention is not limited to the specific devices, methods, conditions, or parameters described and/or shown herein, and that the terminology used herein is for the purpose of describing particular embodiments by way of example only. Thus, the terminology is intended to be broadly construed and is not intended to be limiting of the claimed invention. In addition, as used in the specification including the appended claims, the singular forms “a,” “an,” and “the” include the plural, plural forms include the singular, and reference to a particular numerical value includes at least that particular value, unless the context clearly dictates otherwise. Furthermore, any methods described herein are not intended to be limited to the sequence of steps described but can be carried out in other sequences, unless expressly stated otherwise herein.

The present invention reduces the sensation of pain perceived by a human or animal subject resulting from a puncturing procedure, by providing methods and devices for producing a sensory distraction that confuses or distracts the nerves at or adjacent the puncture site just prior to or simultaneously with the puncturing procedure. For example, a sensory distraction such as an impact, vibration, acoustical signal, or electrical stimulation may be imparted at or adjacent the puncture site just prior to or simultaneously with the puncturing procedure to distract the subject and lessen the perceived sensation of pain from the puncturing procedure itself. The sensory distraction may be imparted by the same device used to carry out the puncturing procedure, or alternatively may be imparted by a separate device used in combination with the device used to carry out the puncturing procedure. Several example devices for implementing the methods of stimulating the puncture site to generate a sensory distraction according to the present invention are described below.

### EXAMPLE 1

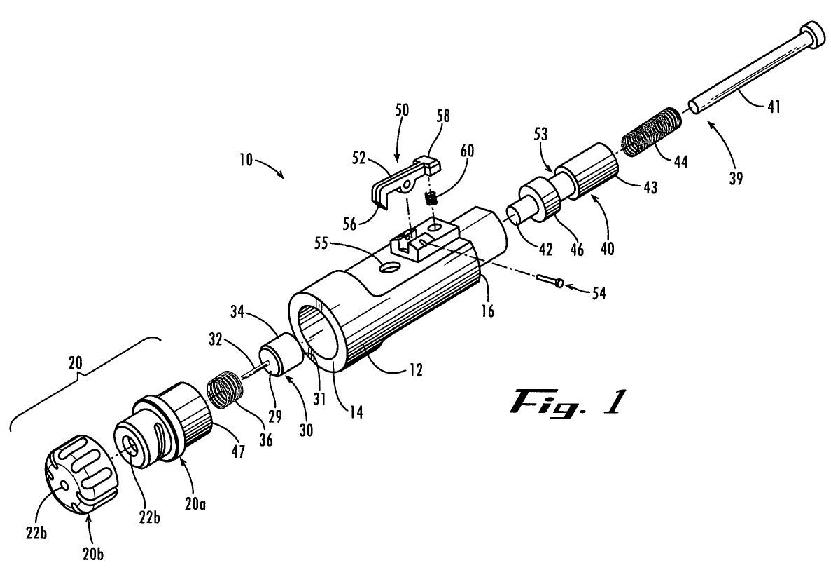

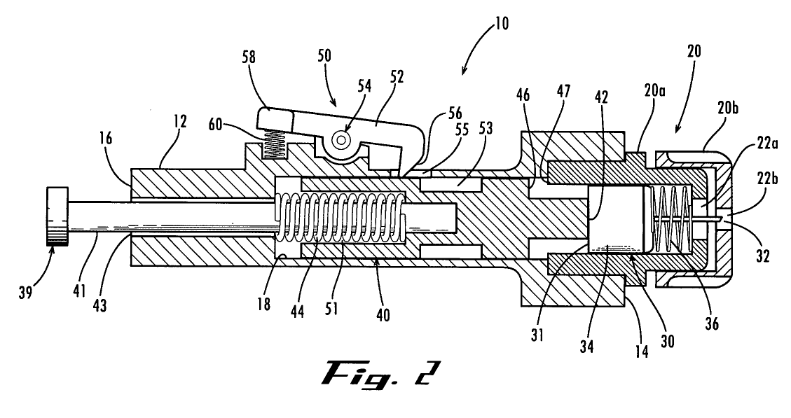

With reference now to FIGS. 1-14, a lancing device 10 according to a first example embodiment of the present invention is shown. FIGS. 1 and 2 show the major components of the lancing device 10, including an outer shell or housing 12. The housing 12 may be, for example, a generally cylindrical element having a proximal end 14 and a distal end 16, and defining an axial interior chamber 18 extending substantially through its length from end to end. The housing 12 is preferably formed of plastic and/or another substantially rigid material(s), for example by injection molding.

The lancing device 10 preferably includes an endcap 20 mounted to the proximal end 14 of the housing. Preferably, the endcap 20 includes an inner cap member 20a and an outer cap member 20b for providing lancing depth adjustment capability, as described in detail with reference to FIGS. 8-14. The endcap 20 cooperatives with the housing 12 to define the axial interior chamber 18. In addition, the endcap 20 preferably includes an opening or passage through which a lancing tip extends for puncturing. For example, the inner and outer caps 20a and 20b may include aligned openings or passages 22a and 22b (collectively, “the opening 22”). Also, depth indicia and/or sample size indicia may be provided on the endcap 20 and/or the housing 12 to indicate the lancing depth setting.

In an alternative embodiment, the endcap is a single piece that is separate from and attached to the housing. And in another alternative embodiment, the endcap is integrally formed with the housing into a single piece without depth adjustment capability, that is, the endcap is essentially an endwall of the housing. As such, the term “endcap” as used herein includes any structure at the proximal end of the housing, whether separate from or integral to the housing.

In still other alternative embodiments, the lancing device enables depth adjustment by other mechanisms. For example, the endcap may have one or more adjustably positionable internal stop members that limit the distance of travel of a lancet, or the endcap may be adjustable so that the axial position of the endcap may be varied to limit the travel. In particular, the endcap can be joined to the housing by a threaded connection permitting the endcap to be extended and retracted axially relative to the housing by twisting the endcap. And in yet other alternative embodiments, a proximal face of the endcap has one or more raised projections or rings for stimulating bloodflow for sample collection, as by pressure and/or pumping, and/or may be transparent or comprise a transparent portion for monitoring sample collection.

A lancet 30 is preferably slidably mounted within the housing 12 and/or the endcap 20, for translation between a retracted position within the housing and endcap, and an extended position wherein a sharp lancing tip portion 32 of the lancet projects through the opening 22 in the endcap 20. A close sliding fit is preferably provided between the lancet 30 and the housing 12 and/or the endcap 20 to minimize lateral movement and rocking of the lancing tip 32 during the lancing operation. The lancet 30 preferably comprises a body portion 34 formed of plastic and/or another material(s), having a proximal end 29 from which the lancing tip 32 projects and a distal end 31 opposite the proximal end. The body portion 34 of the lancet 30 is preferably larger in at least one dimension than the opening 22 to prevent the lancet from passing through the opening and being discharged from the housing 12 and endcap 20. For example, the lancet 30 may be provided with the proximal end 29 of the body portion 34 dimensioned so that it will not pass through the endcap opening 22, but will instead engage the endcap 20 to limit the travel of the lancet. The lancet 30 may be substantially smaller and lighter in weight than standard known lancets, because the lancet does not need to incorporate a gripping surface or engagement features for coupling the lancet with the drive mechanism of the lancing device 10. A return spring 36 is preferably engaged between the lancet 30 and the endcap 20 or housing 12 to retract the lancing tip 32 back into a shielded position after lancing, enclosed within the housing and endcap, to prevent inadvertent needlesticks or bloodborne contamination.

A drive mechanism includes a drive member such as a ram or piston 40 that is preferably mounted to translate axially within the chamber 18 of the housing 12 and/or endcap 20. The piston 40 preferably includes a proximal end 42 for impacting the distal end 31 of the lancet 30 upon firing to propel the lancet into its extended or lancing position, for puncturing the skin of the subject at the lancing site. The contacting surfaces of the piston 40 and the lancet 30 are preferably smooth, parallel surfaces, or provide point contact at the approximate centerpoint of the lancet, to minimize or prevent lateral movement or rocking of the lancet during the lancing procedure. The piston 40 is preferably sized and shaped to generally conform to the inner shaped of the axial interior chamber 18 of the housing 12 and/or endcap 20 with a nice fit for preventing or eliminating lateral movement. In a typical commercial embodiment, for example, the piston 40 and the axial interior chamber 18 are generally cylindrical. In addition, the center of mass of the piston 40 is preferably axially aligned with the center of mass of the lancet 30, further minimizing lateral movement or rocking of the lancet. And the mass of the piston 40 is preferably greater than or equal to that of the lancet 30, which reduces the mass of the components impacting and piercing the skin, with the spring selected to have a greater spring factor to achieve the same lancet tip velocity and force at impact.

A cocking mechanism 39 preferably includes an arm or rod 41 that extends from the piston 40 to a position external of the housing 12, for example through an opening 43 in the distal end 16 of the housing 12 as shown, or alternatively through a side or other opening formed in the housing. The cocking rod 41 may be a separate component affixed or coupled to the piston 40. For example, the cocking rod 41 may be fixedly received in an opening 51 in the piston 40, as shown. Alternatively, the cocking rod 41 may be integrally formed with the piston 40 as a single component.

The drive mechanism preferably further includes a drive spring 44 for driving the piston 40 from a retracted position to an extended position for impacting the lancet 30. The drive spring 44 may be engaged between the piston 40 and the housing 12, for example received in the piston opening 51 as shown, or between the cocking rod 42 and the housing. With the drive spring 44 received in the opening 51 of the piston 40, the proximal end of the drive spring is forward (closer to the lancet) of the distal end of the piston so that it is not “pushing” the piston from behind, thereby helping to reduce any lateral movement of the piston in the chamber 18 and generally providing improved guidance and control. It will be understood that the lancing device 10 may be alternatively provided with a different cocking mechanism, or without a cocking mechanism for single-use lancing devices. In addition, it should be noted that the piston 40 and the drive spring 44 are shown in FIG. 1 exploded to the distal end of the lancing device for convenience of illustration, but that they are actually loaded into the housing from the proximal end.

The piston 40 preferably comprises a flared shoulder, projection, or segment 46 having an expanded dimension for contacting a stop or limit member to limit the travel of the piston during the lancing operation. For example, the stop or limit member may be provided by a distal face portion 47 of the endcap 20, or alternatively by an interference member projecting from another part of the housing 12 or endcap 20, or by another component of the lancing device 12. In addition, the piston 40 has another flared shoulder, projection, or segment 43, with the opening 51 preferably defined therein. The flared segments 43 and 46 of the piston 40 together define a locking notch 53. The locking notch 53 may be a circumferential channel, as shown, or it may be a circular hole or otherwise configured notch.

The lancing device 10 preferably further comprises a trigger mechanism 50. For example, in the depicted embodiment, a rocker arm 52 is pivotally mounted to the housing 12 by a hinge or other pivotal connection 54 approximately midway along the length of the rocker arm. A latch 56 extends from a proximal end of the rocker arm 52, and through an opening 55 in the sidewall of the housing 12 to engage and release the piston 40 during cocking and firing. A release button or contact surface 58 is provided on the distal end of the rocker arm 52, for selectively releasing the trigger mechanism when the user is ready to fire the device and carry out a lancing sequence. A spring 60 normally biases the latch end of the rocker arm 52 inward for engagement with a shoulder defined by the notch 53 of the piston 40 during cocking, until the user overcomes the biasing force of the spring by applying finger pressure onto the release button 58 to pivot the latch 56 out of engagement with the lock notch to fire the lancing device 10. It will be understood that the lancing device 10 may be alternatively provided with a different trigger mechanism.

FIGS. 2-7 depict a sequence of operation of the lancing device 10 and a first method of lancing according to an example embodiment of the present invention. The lancing device 10 is initially delivered to the user in an uncocked, rest position, as shown in FIG. 2. The opening 22 in the endcap 20 may initially be sealed for sterility, as by a penetrable foil covering, or by a plug or cap that is removed by the user prior to use.

The user cocks the device 10 to the cocked or armed position of FIG. 3 by grasping the cocking rod 41 and drawing it away from the housing 12 in the distal direction indicated by direction arrow “d.” The cocking rod 41 preferably includes a knob 62 or other gripping surface to make cocking easier. As the cocking rod 41 is drawn back, the piston 40 is also retracted axially through the chamber 18 of the housing 12, compressing and energizing the drive-spring 44, until the latch 56 of the rocker arm 52 engages the notch 53 of the piston 40 to retain the piston in the cocked or armed position, as shown in FIG. 3.

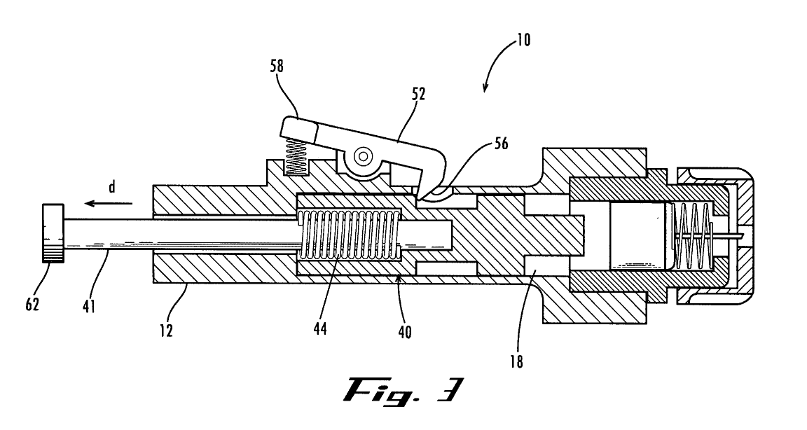

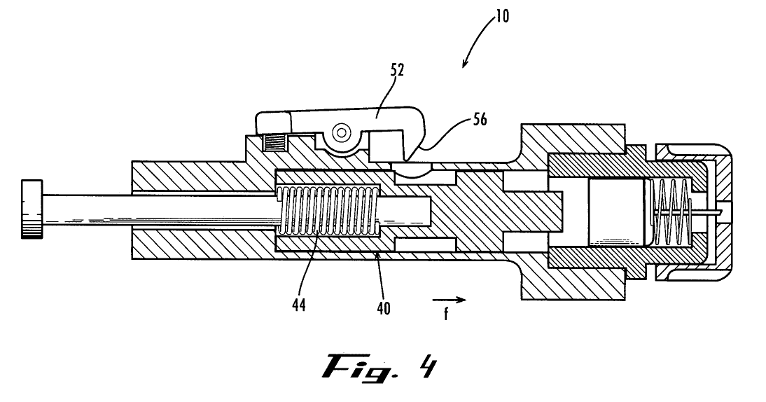

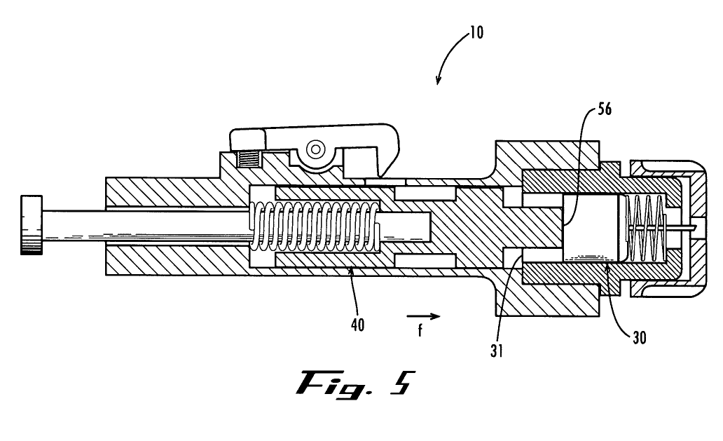

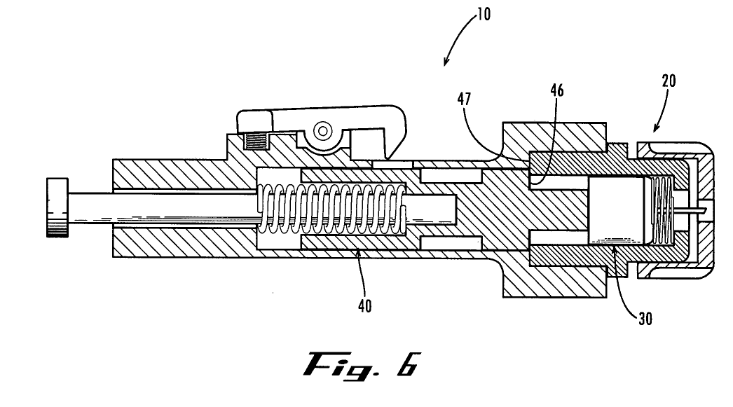

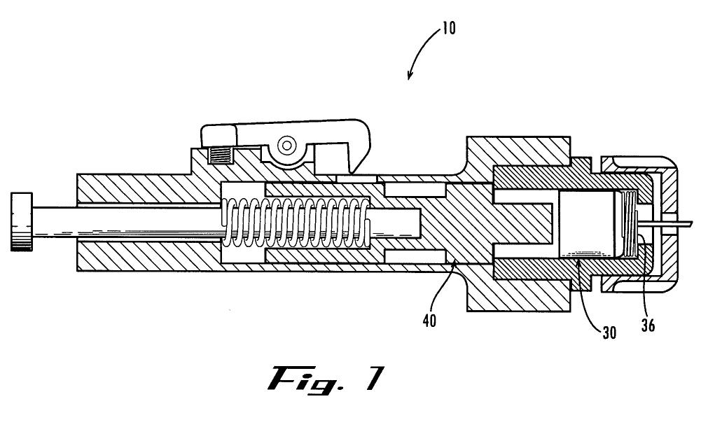

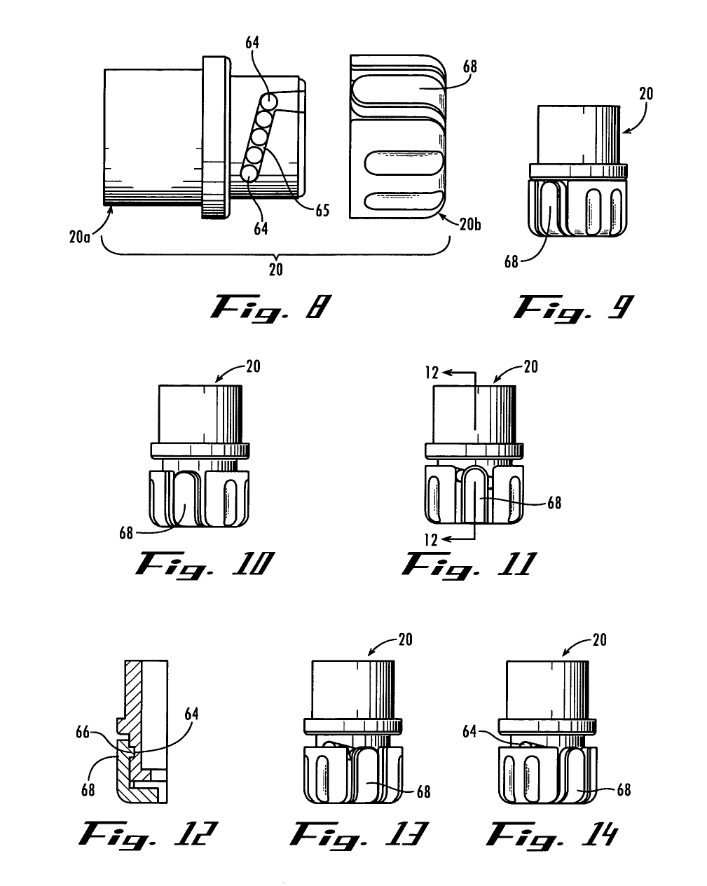

The user then places the proximal face of the lancing device 10 into contact with the skin at the sampling site and presses the release button 58. This draws the latch 56 out of engagement with the piston 40, releasing the piston to be driven by the drive spring 44 in the firing direction indicated by direction arrow “f” in FIG. 4. The proximal end 42 of the piston 40 then impacts the distal end 31 of the lancet 30, as shown in FIG. 5, and propels the lancet 30 in the firing direction “f.” So just before the piston 40 impacts the lancet 30, the piston has kinetic energy and the lancet does not.

After the piston 40 impacts the lancet 30, the expanded segment 46 of the piston contacts the distal face 47 of the endcap 20 to stop the travel of the piston, as shown in FIG. 6. So just after the piston 40 impacts the stop 47, the lancet has kinetic energy and the piston does not. The lancet 30 is thereby decoupled from the piston 40 as the lancet continues on until it is stopped by a stop or limit member in its extended or lancing position, as shown in FIG. 7. The lancet stop may be defined by the inside wall of the endcap or another structure. In any case, the lancet stop and the drive member stop preferably are two separate structures, that is, they are not one and the same (even though they may both be defined by the endcap or the housing or another component of the lancing device).

When the expanded segment 46 of the piston 40 contacts the distal face 47 of the endcap 20 to stop the travel of the piston, a vibration or impact results. This vibration is transmitted through the material of the endcap 20 to the skin at and around the lancing site. The housing 12 and/or the endcap 20 may include a material such as a metal selected for maximizing the transmission of this vibration therethrough. For example, the material may be provided in the housing 12 itself (e.g., a metal housing), in a liner or casing for the housing, and/or in one or more longitudinal strips formed into or attached to the housing surface. In addition, the endcap may be configured for enhanced transfer of the vibration, such as by providing an annular contact surface (around the opening for the lancet tip) to spread the transferred vibration over a large area and/or by providing a few protruding members to concentrate the vibratory effect.

Preferably, the piston stop member is defined by the housing or endcap, as in the distal face 47 of the endcap 20 of the depicted embodiment, which minimizes impediments to the vibratory transfer and thereby maximizes the sensory distraction effect. These impacts are sensed by the subject beginning just prior to the puncturing of the skin by the lancet tip 32, causing a degree of nerve distraction or confusion. It has been discovered that many subjects perceive less pain sensation resulting from the puncturing of the skin when subjected to such a sensory distraction in this manner.

Turning now to FIGS. 8-14, there are shown additional details of the inner and outer caps 20a and 20b of the endcap 20. As shown in FIGS. 8 and 12, the inner cap 20a has a helical channel 65 with a series of recesses 64 that receive a protrusion 66 on a flexible arm 68 of the outer cap 20b. As the user rotates the outer cap 20b, the protrusion 66 on the flexible arm 68 rides in the helical channel 65, thereby moving the endcap 20 axially and changing the penetration depth. The protrusion 66 seats in the recesses 64 to provide discrete depth settings, and the flexible arm 68 deflects when the protrusion is between the recesses 64 to permit adjusting between the depth settings. Any number of depth settings may be provided, space permitting. In the embodiment shown, there are five recesses 64, which provide five discrete depth settings. FIGS. 9, 10, 11, 13 and 14 show these five depth settings, progressing from the deepest to the shallowest.

To replace the lancet 30 in the lancing device 10, the user removes the endcap 20 from the housing 12, with the return spring 36 preferably retained in the endcap. The used lancet 30 is then removed and a fresh one inserted. To do this, the user can grasp the lancet body 34, instead of the lancet tip 32, and insert the lancet 30 into the endcap 20 tip-first. Then the endcap 20, now holding the lancet 30 and the return spring 36, is replaced on the housing 12, ands the lancing device is ready for another use. The process can be repeated as needed for subsequent lancings.

In alternative embodiments, the decoupled drive member is provided by a spring-biased rocker, hammer, or punch, or a transversely driven cam surface, instead of the piston. In another alternative embodiment, the piston stop member extends from the housing, instead of the endcap, to retain the piston in the housing when the endcap is removed for lancet replacement. And in another alternative embodiment, the piston stop member is located closer to the proximal end of the lancing device, and the shoulder of the piston that engages the stop member is defined by the proximal face of the drive member, so that substantially the entire length of the piston is in sliding contact in the axial chamber to minimize lateral movement.

Accordingly, it can be seen that the first example embodiment provides unique pain-reducing features including the vibratory sensory distraction from stopping the drive member before the lancet and the adjustable endcap for setting the lancing penetration depth. Preferably, these features are included together in a single device. It will be understood, however, that these features may be provided in separate devices independent of each other, or combined with other features described herein or elsewhere, as may be desired in particular applications.

### EXAMPLE 2

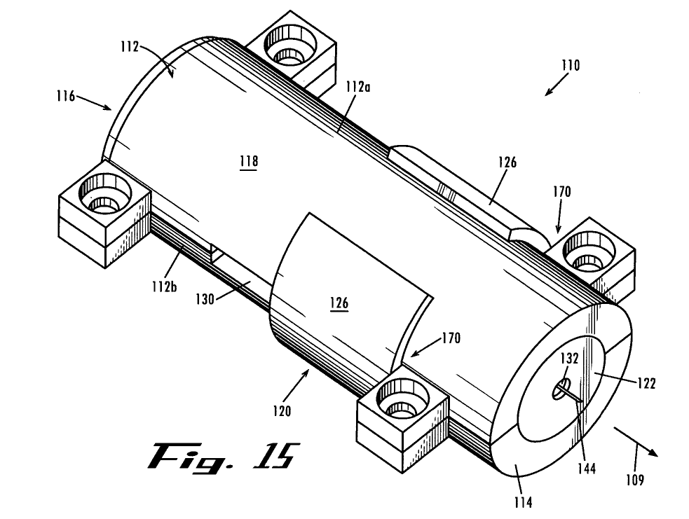

FIGS. 15-18 show a lancing device 110 according to a second example embodiment of the invention, the device also capable of generating a sensory distraction. Referring to FIGS. 15 and 16, the device 110 includes a housing 112, preferably formed of two inter-engaging half-shells 112a, 112b. Generally, the housing 112 has a proximal end 114, a distal end 116, and one or more sidewalls 118. In the depicted embodiment, the housing 112 is generally cylindrical and is formed of plastic and/or other substantially rigid material(s), as for example by injection molding. It will be understood that other housing configurations and materials may be suitably used. In addition, a penetrable foil covering or removable cap (unshown) preferably initially covers the opening at the proximal end 114 of the housing 112 to maintain sterility prior to use of the device 110.

A drive mechanism includes a drive member such as a carrier 120 that is slidably mounted to the housing 112. The carrier 120 preferably includes a carriage 122 mounted within a channel 124 extending axially through the housing 112, and one or more sleeves or wings 126 extending outwardly of the housing 112 and connected to the carriage by a strut 128 projecting through a slot 130 in the sidewall 118 of the housing. The carriage 122 is preferably engaged within the channel 124 with a close sliding fit to minimize rocking and lateral motion, and to constrain the carriage to translation along an axial path parallel to direction arrow “a.” The struts 128 preferably slide within the slot 130 with a close sliding fit, further defining the path of translation of the carrier 120 and preventing twisting of the carrier within the housing 112. The sleeves or wings 126 provide a gripping surface for the user to grasp to pull back the carrier 120 for cocking the lancing device. As such, the wings 126 may be provided by tabs, collars, finger rests, and other grasping members. In the depicted embodiment, for example, the wings 126 generally conform to the shape of the housing 112 and are in the form of sleeves that extend a majority of the way around the housing.

In addition, a proximal hole or opening 132 is formed in the proximal face of the carriage 122 of the carrier 120 for allowing passage of a lancet tip during lancing, as described below. A distal hole or opening 134 is formed in the distal face of the carriage 122 of the carrier 120 for allowing passage through it of a cocking arm portion of the lancet sled, as described below. And a stepped bore 136 (including a track, channel, etc.) preferably extends axially through the carriage 122 of the carrier 120, forming a distally-facing shoulder 138.

A lancet sled 140 is slidably mounted within the bore 136 of the carriage 122, and includes a lancet 142 having a sharp lancing tip 144. The bore 136 is preferably cylindrical, but it may have a square of other cross-sectional shape, if desired, and is axially longer than a body of the lancet 142. The lancet 142 may be integrally formed with the lancet sled 140, for example, in a disposable lancing device embodiment. Or it may be a separate component secured to the sled, as by a friction fit within a receptacle of the sled as shown, for example, in a multi-use lancing device embodiment. A return spring 146 is preferably engaged between a flange or projection 147 extending from the lancet sled, and the shoulder 138 of the carrier 120.

A cocking mechanism preferably comprises at least one cocking arm 148 that extends distally from the lancet sled 140 and includes a catch such as a barb 150 for engaging an engagement surface such as a flange or shoulder 152 of the housing 112 to secure the sled and carrier arrangement in a cocked position, as shown in FIG. 16. Alternatively, the cocking arm 148 may extend from the carrier 120, from the lancet 42 (e.g., in embodiments with the lancet and sled integrally formed as one piece), or from another component of the device 110. And the drive mechanism includes a drive spring 154 that is preferably engaged between the carrier 120 and the housing 112, for driving the lancet sled 140 and carrier 120 through a lancing stroke from the cocked position (see FIG. 16) to the extended position (see FIG. 18). It will be understood that other conventional cocking mechanisms may be suitably employed.

A trigger mechanism includes a trigger or release button 160 that is preferably mounted at the distal end 116 of the housing 112. The release button 160 includes a catch release member such as an inclined cam face 162 for engagement against a cooperating inclined face 163 or other catch release member of the cocking arm 148. A spring 164 is preferably provided to bias the release button 160 distally from the housing 112. When the release button 160 is depressed, the inclined cam face 162 engages the cooperating inclined face 163 to release the barb 150 from the shoulder 152, thereby firing the device 110 and initiating a lancing operation. It will be understood that other conventional trigger mechanisms may be suitably employed.

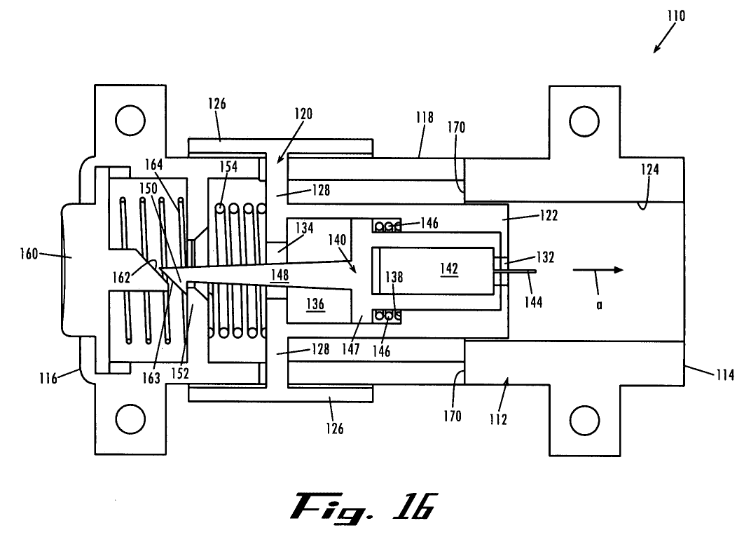

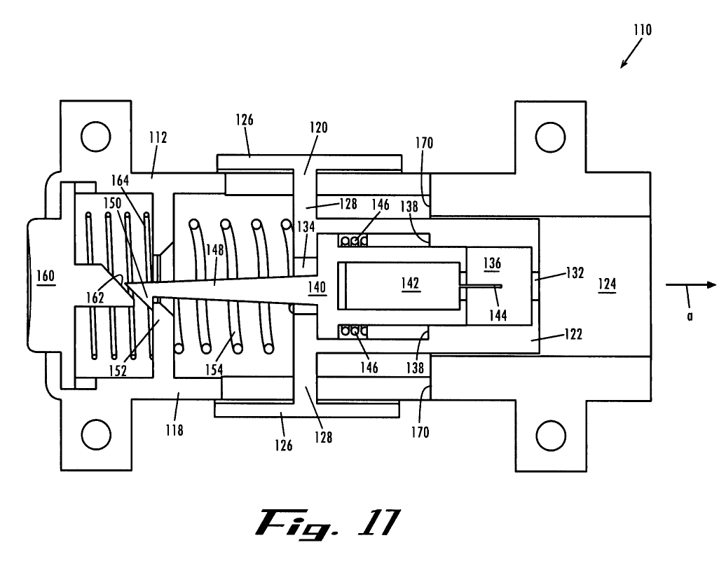

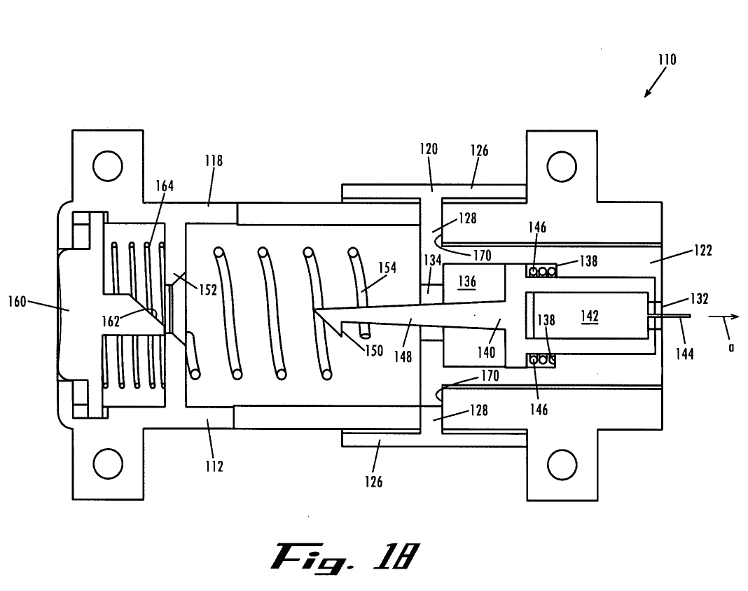

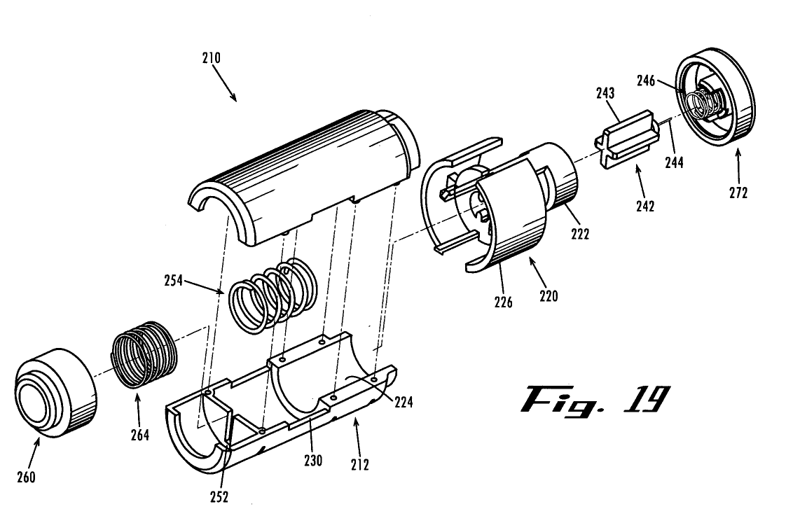

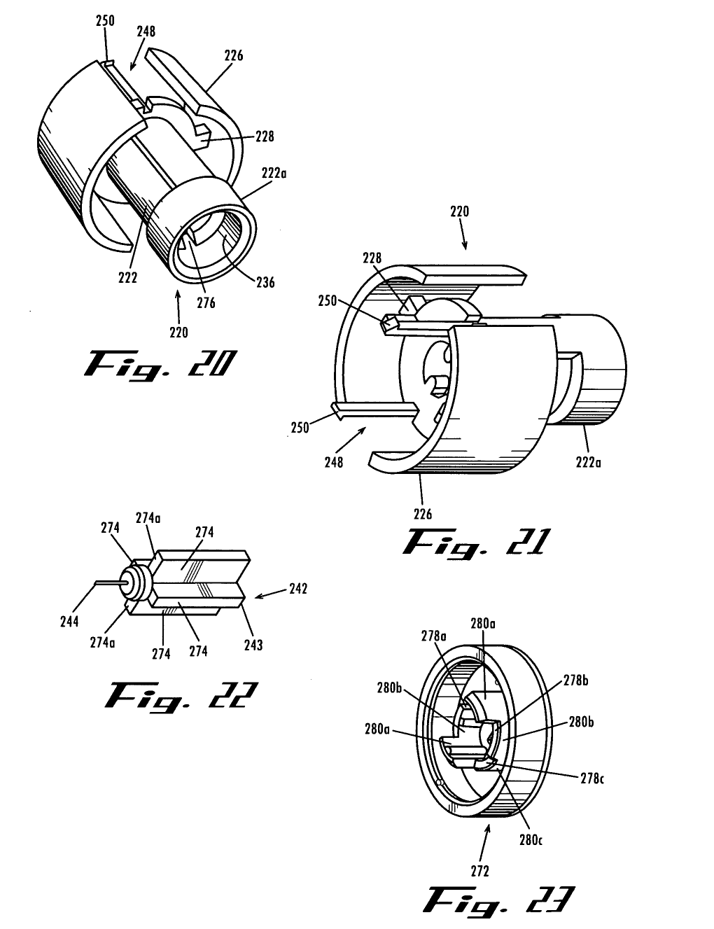

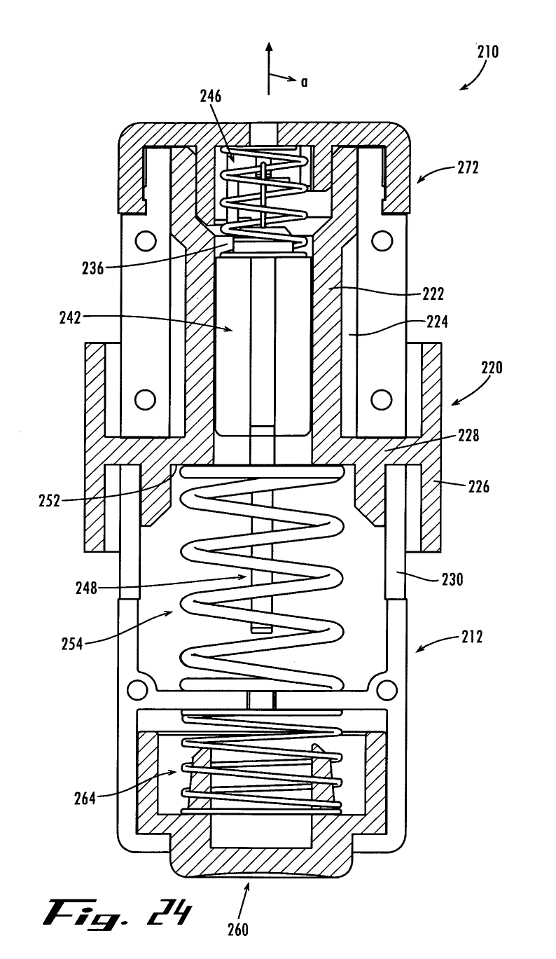

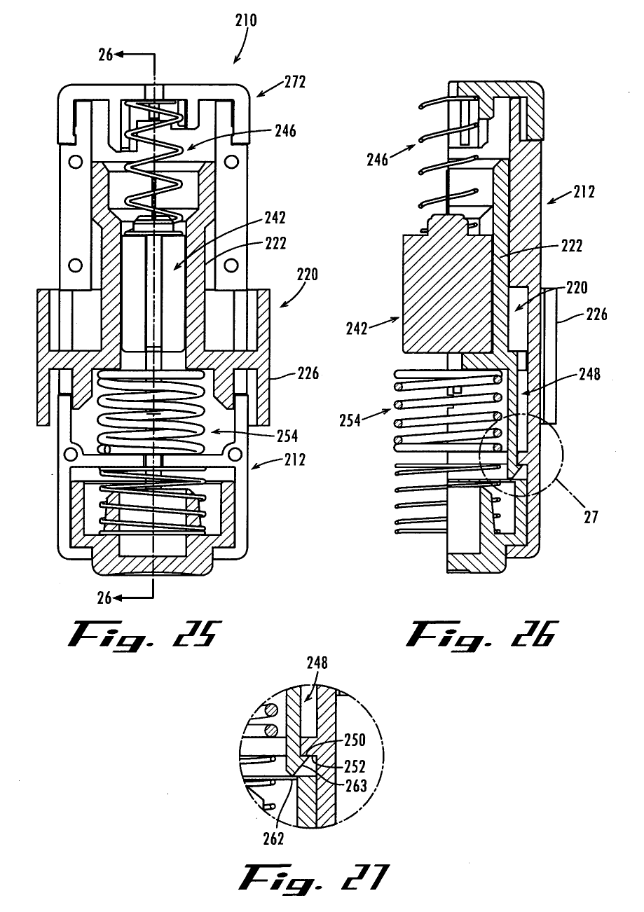

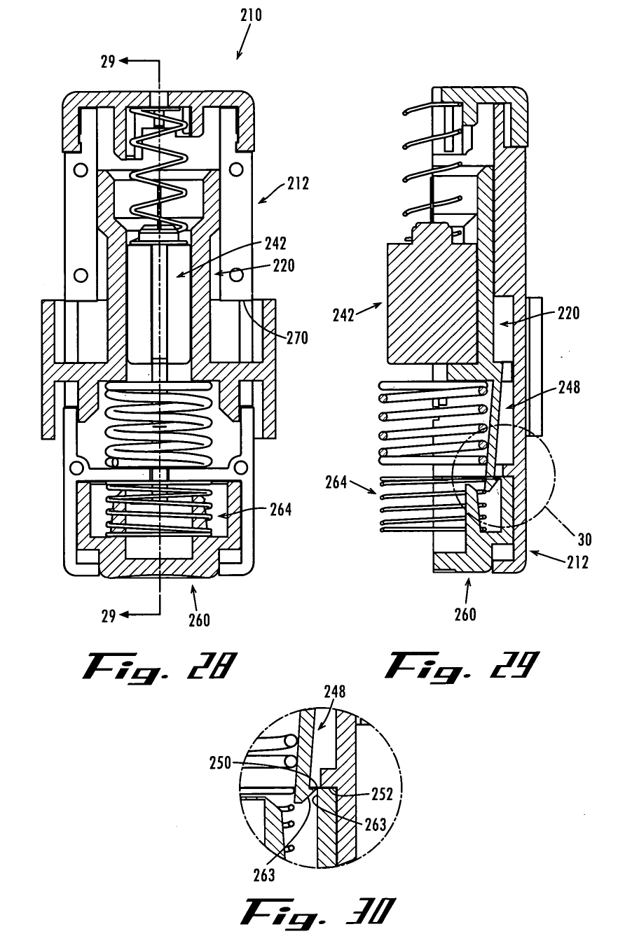

FIGS. 16-18 depict a sequence of operation of the lancing device 110 and a second example method of lancing according to the present invention. The lancing device 110 is preferably delivered to the user in an uncocked state, with the drive spring 154 substantially or partially relaxed. The user cocks the device 110 by gripping the housing 112 and the wings 126, and pulling the wings (and thereby the carriage 122 and sled 140) distally relative to the housing, into the cocked position shown in FIG. 16, with the drive spring 154 substantially fully compressed. Interaction between the shoulder 138 of the carriage 122 and the flange 147 of the sled 140 withdraws the sled distally along with the carrier during cocking. Engagement of the catch barb 150 with the engagement shoulder 152 retains the carrier 120 in a cocked position after the wings 126 are released, allowing the carrier to move forward (proximally) within the channel 124 under the biasing influence of the drive spring 154, as shown in FIG. 17. The user then places the proximal face 114 of the housing 112 against the skin at the sampling site of a human or animal subject. The device 110 is fired to complete the lancing operation by pressing the release button 160, the inclined face 162 of which contacts the cooperating inclined face 163 of the cocking arm 148 and disengages the catch barb 150 from the engagement shoulder 152. Then the drive spring 154 propels the sled carrier 120 and the lancet sled 140 forward to initiate the lancing stroke.

When the lancing stroke begins, the sled 140 is retained in a retracted position, toward the distal end of the bore 136 in the carriage 122. The sled 140 is retracted in the back of the bore 136 as a result of the engagement of the barb 150 and the shoulder 152 holding the carrier back, combined with the forward bias of the drive spring 154 against the carrier 120 (see FIG. 17). After operating the trigger to fire the device 110, the carrier 120 is released and now moves forward under the influence of the drive spring 154, carrying the lancet sled 140 along with it.

Next, the carrier 120 and lancet sled 140 move forward together until there is an impact with a carrier stop such as a proximal endwall 170 of the slot 130 of the housing 112. For example, the stopping may be caused by the carrier struts 128 impacting the housing slot proximal endwalls 170, the carrier wings 126 impacting a protruding structure on the exterior of the housing 112, or by other means. Inertia propels the lancet sled 140 forward after the carrier 120 stops, with the sled 140 and lancet 142 no longer being coupled to the carrier 120 or the spring 144 of the drive mechanism.

The sled 140 slides forward through the bore 136 to the extended position, shown in FIGS. 15 and 18, wherein the sharp lancet tip 144 passes through the hole 132 and projects a distance beyond the proximal face of the housing 112 to puncture the subject's skin at the sampling site. The lancet sled 140 is stopped in the extended position by a stop member such as an inside wall of the carriage, the housing, an endcap, or another structure. In any case, the lancet sled stop and the carrier stop preferably are two separate structures, that is, they are not one and the same (even though they may both be defined by the endcap or the housing or another component of the lancing device).

When the carrier 120 impacts the carrier stop 170, a vibration or impact results. This vibration is transmitted through the material of the housing 112 to the skin at and around the lancing site. Preferably, the carrier stop member is defined by the housing 112, as in the depicted embodiment, which minimizes impediments to the vibratory transfer and thereby maximizes the sensory distraction effect. In addition, the housing 112 may include a material such as a metal selected for maximizing the transmission of this vibration therethrough. In any event, the vibration is sensed by the subject just prior to the puncturing of the skin by the lancet tip 144, causing a degree of nerve distraction or confusion. It has been discovered that many subjects perceive less pain sensation resulting from the puncturing of the skin when subjected to a sensory distraction in this manner.

In alternative embodiments, a penetration-depth adjustment mechanism is provided to allow adjustment of the depth of penetration of the lancet tip into the skin of the sampling site. For example, the penetration-depth adjustment mechanism may be provided by a rotatable endcap on the proximal end of the housing, with the endcap joined to the housing by a threaded connection permitting the endcap to be extended and retracted axially relative to the housing by twisting the endcap. In still other alternative embodiments, the endcap has one or more adjustably positionable internal stop members that limit the distance of travel of a lancet.

Accordingly, it can be seen that the second example embodiment provides unique pain-reducing features including the vibratory sensory distraction from stopping the carrier before the lancet and the adjustable endcap for setting the lancing penetration depth. Preferably, these features are included together in a single device. It will be understood, however, that these features may be provided in separate devices independent of each other, or combined with other features described herein or elsewhere, as may be desired in particular applications.

### EXAMPLE 3

FIGS. 19-40 show a lancing device 210 according to a third example embodiment of the invention, the device also capable of generating a sensory distraction. The lancing device 210 is similar to the lancing device 110 of the second example embodiment. Structural differences include the device 210 combining the carrier and sled into one component, and the addition of an innovative penetration depth adjustment mechanism, as described below.

Referring to FIGS. 19-24, the lancing device 210 includes a housing 212, a drive mechanism, a lancet 242, a cocking mechanism, a trigger mechanism, and an endcap 272. Generally, the housing 212 has a proximal end, a distal end, and one or more sidewalls. The endcap 272 preferably includes an opening or passage through which a lancing tip extends for puncturing. In the depicted embodiment, the endcap is separate from and attached to the housing. Alternatively, the endcap may be integrally formed with the housing into a single piece without depth adjustment capability, in which case the endcap is essentially an endwall of the housing.

The drive mechanism includes a lancet carrier 120, shown with particularity in FIGS. 20 and 21. The lancet carrier 220 preferably includes a carriage 222 mounted within a chamber 224 extending axially through the housing 212, and one or more sleeves or wings 226 extending outwardly of the housing and connected to the carriage by a strut 228 projecting through a slot 230 in the housing. The carriage 222 translations along an axial path parallel to direction arrow “a.” A proximal hole or opening is formed at the proximal end of the carriage 222 of the carrier 220 for allowing passage of a lancet tip during lancing, as described below. A bore 236 preferably extends axially through the carriage 222 of the carrier 220. And the drive mechanism further includes a drive spring 254 that is preferably engaged between the carrier 220 and the housing 212, for driving the carrier and lancet 242 through a lancing stroke from the cocked position (see FIG. 25) to the extended position (see FIG. 32).

The lancet 242 is preferably slidably mounted within the bore 236 of the carriage 222, and includes a lancet body 243 and a sharp lancing tip 244. The bore 236 is preferably cylindrical, but it may have a square of other cross-sectional shape, if desired, and is axially longer than the lancet body 243. Alternatively, the carriage 222 may be provided by a disc, piston, finger, or other drive member that pushes the lancet 242, but that does not have a bore for receiving it, so that the lancet slidingly floats relative to the carriage and in the housing chamber 224. The lancet 242 may be of a conventional type, or it may have special features for cooperating with the endcap to control the penetration depth, as described below. A return spring 246 is preferably engaged between the lancet body 243 and the endcap 272.

The cocking mechanism preferably comprises at least one cocking arm and engagement surface for securing the carrier in a cocked position. In the depicted embodiment, for example, the cocking mechanism has two cocking arms 248 that extend distally from the carrier 240, each with a catch such as a barb 250 for engaging an engagement surface such as a flange or shoulder 252 of the housing 212, as shown in FIG. 27.

The trigger mechanism preferably comprises a trigger spring 264 and a release button 260. The release button 260 preferably includes a catch release member such as an inclined cam face 262 for engagement against a cooperating inclined face 263 or other catch release member of the cocking arm 248, as shown in FIG. 30.

In alternative embodiments, the carrier 220 is included in other lancing devices in which the lancet fits snugly therein without slidably floating therein so that the lancet and carrier do not decouple during the lancing stroke. In this way, the carriage of the carrier acts as a conventional lancet holder. Such lancing devices may include the cocking and trigger mechanisms described herein or others. While these lancing devices do not produce the pain-reducing advantages associated with decoupling the lancet from the drive mechanism, they nevertheless provide improved guidance and control of the lancet, which tends to reduce lateral movement and rocking of the lancet and thereby reduce pain sensed during puncturing.

FIGS. 24-32 depict a sequence of operation of the lancing device 210 and a third example method of lancing according to the present invention. As shown in FIG. 24, the lancing device 210 is preferably delivered to the user in an uncocked, rest state, with the drive spring 254 substantially or partially relaxed. The user cocks the device 210 by gripping the housing 212 and the wings 226, and pulling the wings (and thereby the carriage 222 and lancet 242) distally relative to the housing, into the cocked position shown in FIGS. 25-27, with the drive spring 254 substantially fully compressed. The return spring 246 withdraws the lancet 242 distally along with the carrier 220 during cocking. Engagement of the cocking arm catch barb 250 with the engagement shoulder 252 retains the carrier 220 in the cocked position after the wings 226 are released, as shown in FIG. 27.

The user then places the proximal face of the housing 212 against the skin at the sampling site of a human or animal subject. The device 210 is fired to complete the lancing operation by pressing the release button 260, as shown in FIGS. 28-30. When this is done, the inclined face 262 of the release button 260 contacts the cooperating inclined face 263 of the cocking arm 248 and disengages the catch barb 250 from the engagement shoulder 252, as shown in FIG. 30. Upon operating the trigger mechanism, the carrier 220 is released and the lancing stroke is initiated. The carrier 220 is now propelled forward under the influence of the drive spring 254, carrying the lancet 242 along with it.

The carrier 220 and lancet 242 move forward together until there is an impact with a carrier stop such as a proximal endwall 270 of the slot 230 in the housing 212. For example, the stopping may be caused by the carrier struts 228 impacting the housing slot proximal endwalls 270 (as shown in FIG. 31), a proximal face of the carriage 222 impacting a distal face of the endcap 272, or by other means. Inertia propels the lancet 242 forward after the carrier 220 stops, with the lancet 242 no longer being coupled to the carrier 220 or the spring 244 of the drive mechanism.

The lancet 242 slides forward through the bore 236 of the carriage 222 to the extended position, shown in FIG. 32. In this position, the sharp lancet tip 244 passes through a hole or opening 232 in the endcap 272 and projects a distance beyond the endcap to puncture the subject's skin at the sampling site.

The lancet 242 is stopped in the extended position by a stop member such as an inside wall of the carriage, the housing, an endcap, or another structure. In any case, the lancet stop and the carrier stop preferably are two separate structures, that is, they are not one and the same (even though they may both be defined by the endcap or the housing or another component of the lancing device).

When the carrier 220 impacts the carrier stop 270, a vibration or impact results. This vibration is transmitted through the material of the housing 212 and endcap 272 to the skin at and around the lancing site. Preferably, the carrier stop member is defined by the housing 212 and/or endcap 272, as in the depicted embodiment, which minimizes impediments to the vibratory transfer and thereby maximizes the sensory distraction effect. In addition, the housing 212 may include a material such as a metal selected for maximizing the transmission of this vibration therethrough. In any event, the vibration is sensed by the subject beginning just prior to the puncturing of the skin by the lancet tip 244, causing a degree of nerve distraction or confusion at the puncture site. It has been discovered that many subjects perceive less pain sensation resulting from the puncturing of the skin when subjected to a sensory distraction in this manner.

In addition to the free-floating lancet and sensory distraction features, a penetration-depth adjustment mechanism may be provided to allow adjustment of the depth of penetration of the lancet tip into the skin of the sampling site. For example, the penetration-depth adjustment mechanism may be provided by a uniquely configured carrier 220, lancet 242, and endcap 272, as shown in FIGS. 20-23 and 33-39. The carrier 220 and the lancet 242 are keyed so that they fit together in a specific orientation. Preferably, the lancet 242 has at least one male key member and the carrier 220 has at least one female key member, or vice versa, that cooperate to properly orient the lancet. In addition, the lancet 242 has at least one contact surface and the endcap 272 has a plurality of stop surfaces, or vice versa, for adjusting the puncturing depth of the lancet tip.

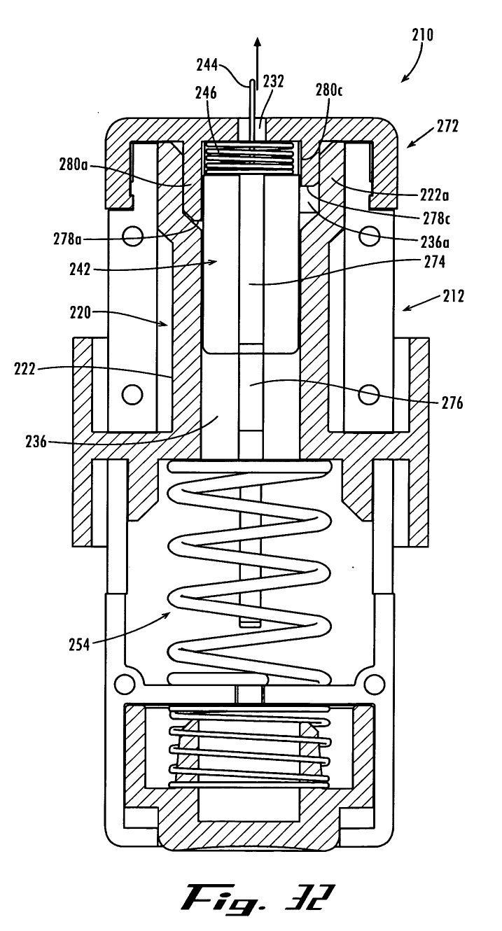

In the depicted embodiment, for example, the lancet body 243 comprises four arms 274 in the shape of a “t” with two opposing ones of the arms having outer portions 274a making them longer than the other two arms (see FIGS. 22 and 39). The two male key members and two contact surfaces are formed by the same structure, namely, the two outer portions 274a of the arms 274 of the lancet body 243. The two arm outer portions 274a are received in two female key channels 276 in the carrier 220 to properly orient the lancet 242 (see FIGS. 20, 32, 33, and 39). And to provide penetration depth adjustability, the two arm outer portions 274a selectively engage two sets of three stop surfaces 278a, 278b, and 278c (collectively, the “stop surfaces 278”) defined by protrusions 280a, 280b, and 280c (collectively, the “protrusions 280”) extending distally from the endcap 272 (see FIGS. 23, 32 and 33). It will be understood that another number of protrusions 280 and stop surfaces 278 may be provided, as may be desired for a given application.

The protrusions 280 are circumferentially arranged relative to the lancet 242, so that the arms 274 without outer portions 274a are circumscribed by the circumferential protrusions, but the outer portions interfere with the protrusions when they are in alignment (see FIG. 39). In this way, the endcap 272 can be rotated so that one of the three protrusions 280 of each protrusion set aligns with and contacts the two outer portions 274a to stop the lancet 242, while the other two protrusions of each set are out of alignment with the outer portions and do not interfere with the lancet. Preferably, the carriage 222 has a flared proximal section 222a with a flared bore 236a that receives the two protrusions 280 that are not aligned with the outer portions 274a of the longer arms 274 (see FIGS. 20, 32, and 39). The flared bore 236a has a larger diameter or other size-indicating dimension than the bore 236.

FIGS. 34-38 show the use of the adjustment mechanism and a method of adjusting a lancing device for various penetration depth settings. In FIG. 34, the endcap 272 is positioned with the first/longest protrusions 280a aligned with the arm outer portions 274a, so that if the lancing device is accidentally fired the lancing tip 244 will not extend beyond the endcap.

In FIG. 35, the endcap 272 has been rotated (in the direction of arrow “r”) to a shallow puncturing depth position with the second/intermediate length protrusions 280b aligned with the arm outer portions 274a. In FIG. 36, the lancet 242 has been fired and is in the puncturing position, with the second/intermediate length protrusions 280b contacting the arm outer portions 274a to stop the lancet in the shallow puncturing depth position.

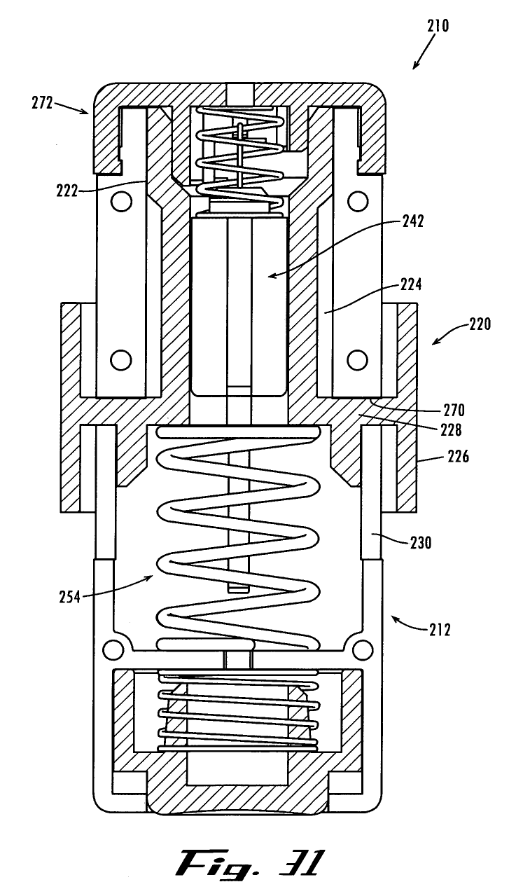

In FIG. 37, the endcap 272 has been rotated farther to a deep puncturing depth position with the third/shortest length protrusions 280c aligned with the arm outer portions 274a. In FIG. 38, the lancet 242 has been fired and is in the puncturing position, with the third/shortest length protrusions 280c contacting the arm outer portions 274a to stop the lancet later, in the deep puncturing depth position.

It will be understood that the adjustment mechanism can be provided in alternative forms. In one alternative embodiment, the carrier 220 and the endcap 272 are the same, but the lancet 342 has a body 343 that generally conforms to the shape of the bore 236 defined in the carriage 222 and that has outer portions 374a extending therefrom (see FIG. 40). In another alternative embodiment, the endcap protrusions that are not aligned with the outer portions are received in recesses in the lancet body to avoid movement-limiting interference with the lancet. And in another alternative embodiment, the adjustment mechanism is included in lancing device with a lancet that is coupled to the drive mechanism so that it does not float separately therefrom, with the female key channels or other structures being defined in the housing or another component of the lancing device.

Accordingly, it can be seen that the third example embodiment provides unique pain-reducing features including the vibratory sensory distraction from stopping the carrier before the lancet and the adjustable endcap for setting the lancing penetration depth. Preferably, these features are included together in a single device. It will be understood, however, that these features may be provided in separate devices independent of each other, or combined with other features described herein or elsewhere, as may be desired in particular applications.

### EXAMPLE 4

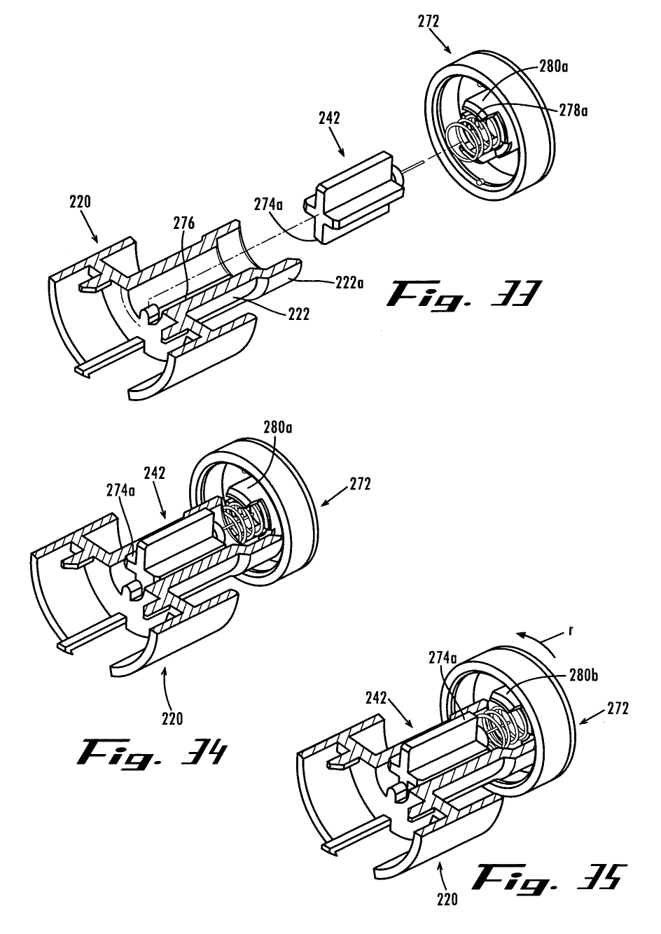

FIGS. 41-62 show a lancing device 410 according to a fourth example embodiment of the invention, the device also capable of generating a sensory distraction. The lancing device 410 is similar to the lancing device 210 of the third example embodiment. Structural differences include an improved penetration depth adjustment mechanism, as described below.

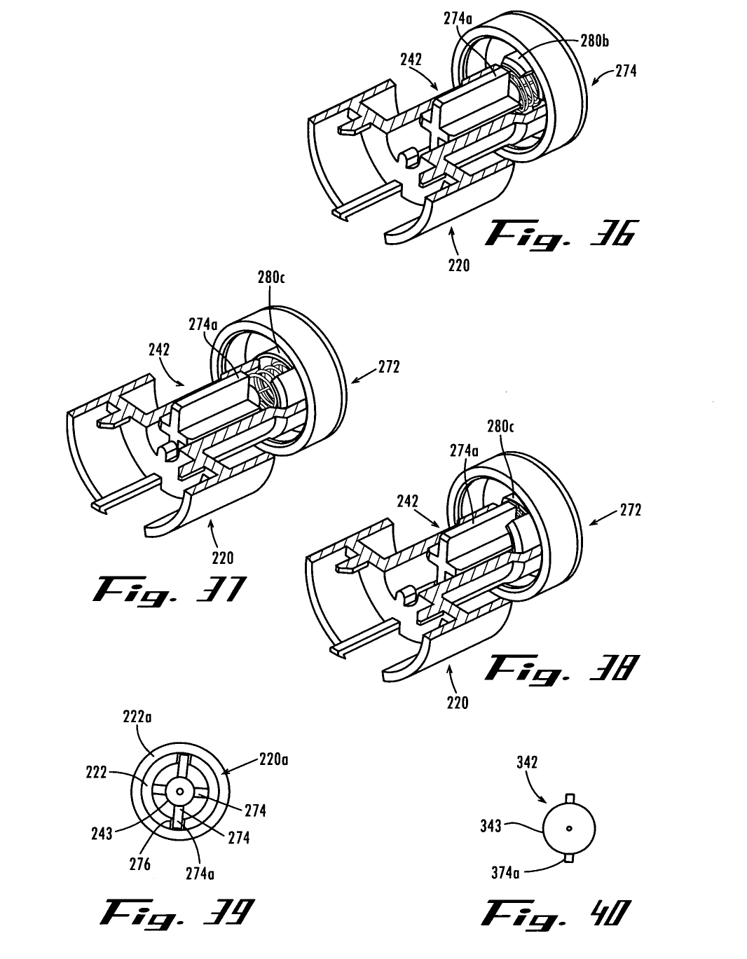

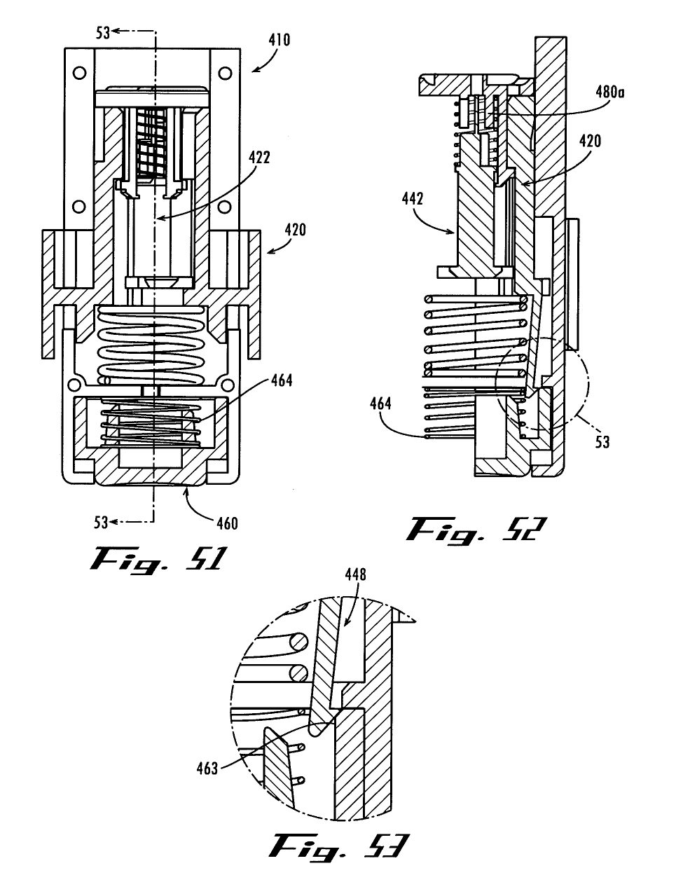

Referring to FIGS. 41-47, the lancing device 410 includes a housing 412, a drive mechanism, a lancet 442, a cocking mechanism, a trigger mechanism, and an endcap 472. The drive mechanism includes a lancet carrier 420, shown with particularity in FIGS. 42A and 42B. The lancet carrier 420 preferably includes a carriage 422 mounted within a chamber 424 extending axially through the housing 412. A bore 436 preferably extends axially through the carriage 422. The drive mechanism further includes a drive spring 454. The lancet 442 includes a lancet body 443 and a sharp lancing tip 444. A return spring 446 is preferably engaged between the lancet body 443 and the endcap 472. The cocking mechanism preferably comprises at least one cocking arm 448 and engagement surface 452 for retaining the carrier 420 in a cocked position, as shown in FIG. 50. The trigger mechanism preferably comprises a trigger spring 464 and a release button 460 with a catch release member 463, as shown in FIG. 53.

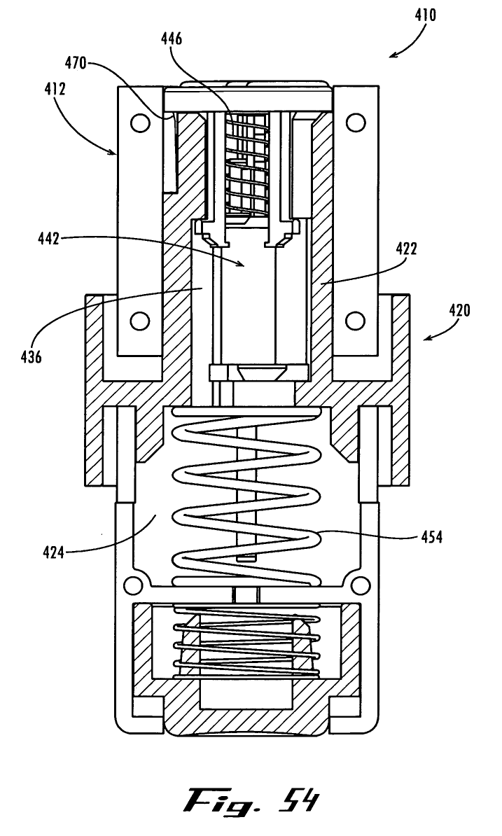

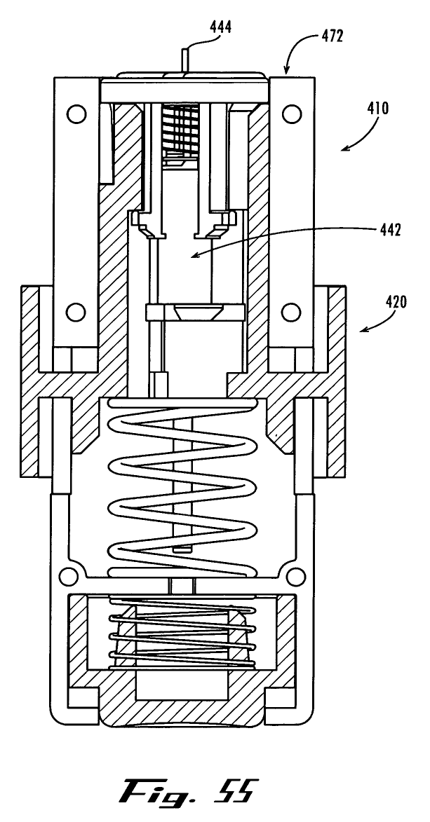

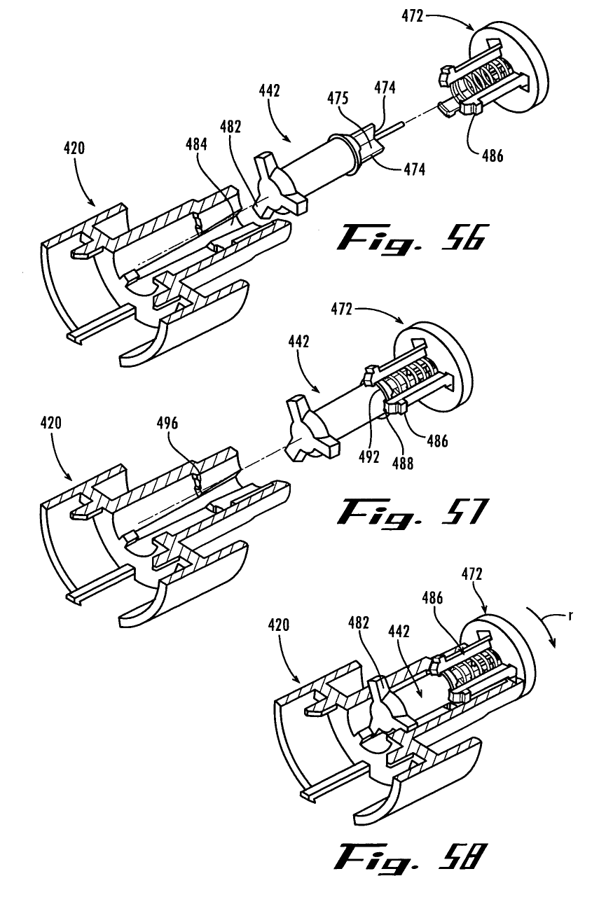

FIGS. 47-55 depict a sequence of operation of the lancing device 410 and a fourth example method of lancing according to the present invention. FIG. 47 shows the lancing device 410 with the carrier 420 and lancet 442 in an uncocked, rest state. FIGS. 48-50 show the lancing device 410 with the carrier 420 and lancet 442 in the cocked position, with the drive spring 454 substantially fully compressed and the cocking arm 448 retained by the engagement surface 452. FIGS. 51-53 show the lancing device 410 with the carrier 420 and lancet 442 in an activated position, with the cocking arm 448 being released by the catch release member 463 of the trigger button 460 to initiate the lancing stroke. FIG. 54 shows the lancing device 410 in the pre-stimulation position with the carrier 420 motion stopped by the carrier stop 470. And FIG. 55 shows the lancing device 410 with the carrier 420 stopped and the lancet 242 having decoupled therefrom and moved forward to the extended position with the sharp lancet tip 244 projecting beyond the endcap 472 to puncture the subject's skin.

A benefit is that, when the carrier 420 impacts the carrier stop 470, a vibration or impact results, similarly to the third embodiment. This vibration is transmitted through the device 410 to the skin at and around the lancing site and sensed by the subject just prior to the puncturing of the skin by the lancet tip, causing a degree of nerve distraction or confusion and reducing the perceived pain from the puncturing.

In addition to the free-floating lancet and sensory distraction features, a penetration-depth adjustment mechanism may be provided to allow adjustment of the depth of penetration of the lancet tip into the skin of the sampling site. For example, the penetration-depth adjustment mechanism may be provided by a uniquely configured carrier 420, lancet 442, and endcap 472, as shown in FIGS. 41-46 and 56-62. The penetration-depth adjustment mechanism of this device 410, the fourth example embodiment, is similar to the penetration-depth adjustment mechanism of the device 210 of the third example embodiment.

The carrier 420 and the lancet 442 are keyed so that they fit together in a specific orientation. Preferably, the lancet 442 has at least one male key member and the carrier 420 has at least one female key member, or vice versa, that cooperate to properly orient the lancet. In the depicted embodiment, for example, the lancet 442 has three male key tabs 482 extending from a distal section 443b of the lancet body and slidingly received in three female key guide slots 484 in the carrier 420 to properly orient the lancet (see FIGS. 42B-44 and 56-58).

In addition, the lancet 442 has at least one contact surface and the endcap 472 has a plurality of stop surfaces, or vice versa, for adjusting the puncturing depth of the lancet tip. In the depicted embodiment, for example, the lancet body 443 comprises a proximal section 443a including three radial arms 474 at equal angles in the shape of a “Y”, with three recesses 475 between the arms (see FIGS. 43 and 56). To provide penetration depth adjustability, the three radial arms 474 selectively engage three sets of three stop surfaces 478a, 478b, and 478c (collectively, the “stop surfaces 478”) defined by protrusions 480a, 480b, and 480c (collectively, the “protrusions 480”) extending distally from the endcap 472 (see FIGS. 46 and 52). Of course, the stop surface 478c can be defined directly on the endcap 472, if desired.

The protrusions 480 are arranged circumferentially and sequentially on the endcap 472 so that all of one set of the protrusions, for example the longest protrusions 480a, align with and engage the contact surfaces on the radial arms 474, while the remaining sets of protrusions align with and are received into the recesses 475 between the radial tabs. In this way, the endcap 472 can be rotated so that one of the three protrusions 480 of each protrusion set aligns with and contacts the radial arms 474 to stop the lancet 442, while the other two protrusions of each set are out of alignment with the radial tabs and do not interfere with the lancet. Accordingly, the number and spacing of protrusions 480 and stop surfaces 478 preferably is the same as the number of radial arms 474. It will be understood that another number and spacing of protrusions 480, stop surfaces 478, and radial arms 474 may be provided, as may be desired for a given application.

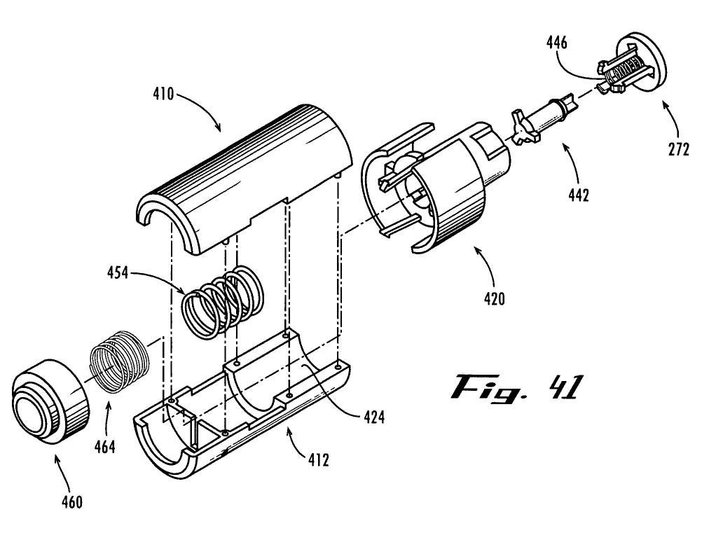

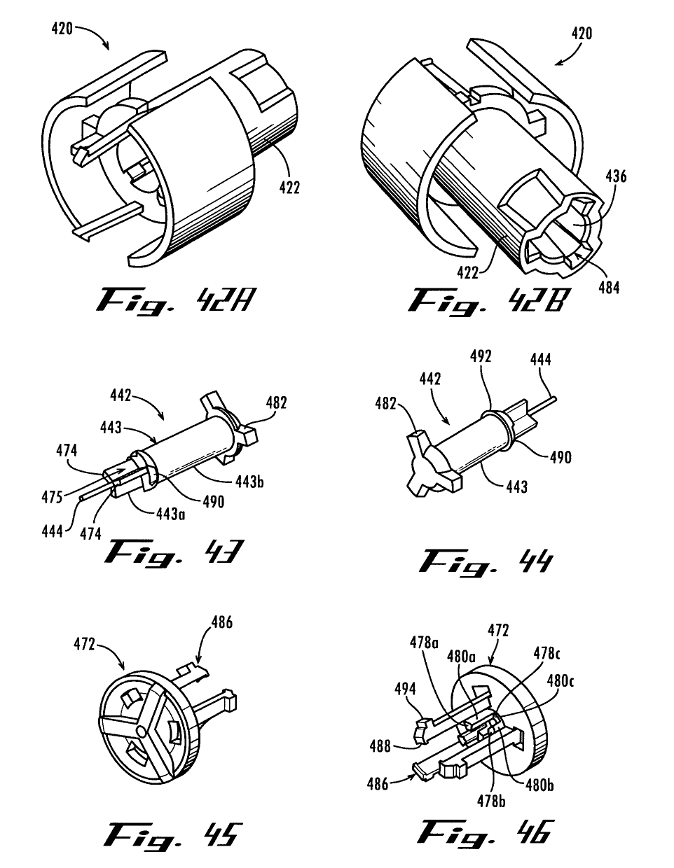

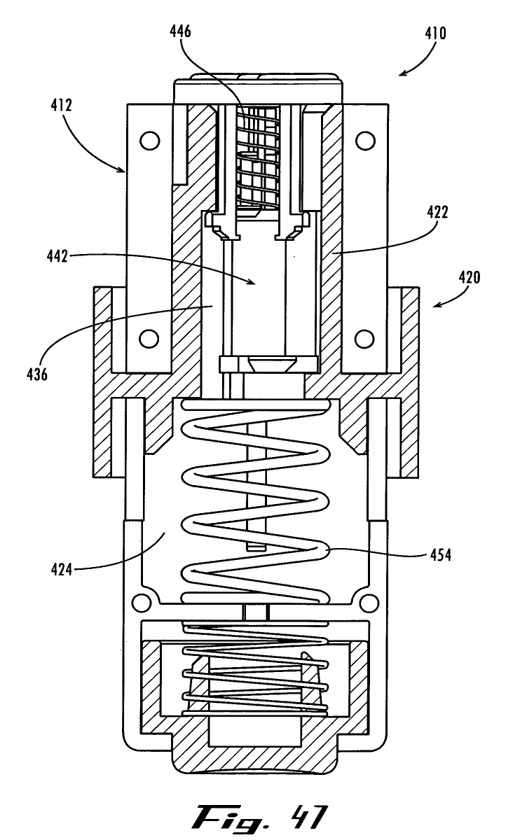

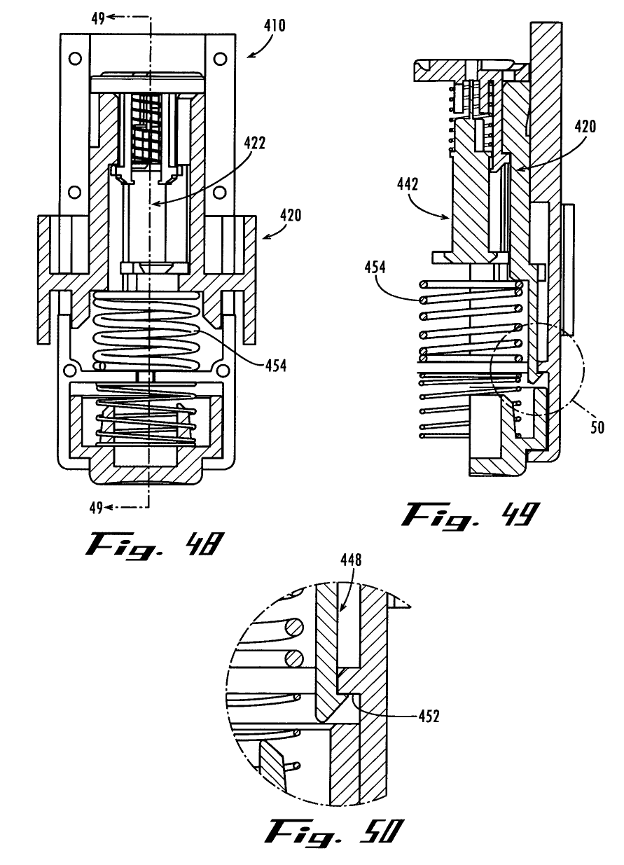

Furthermore, the lancing device 410 includes keepers for retaining the endcap 472 and the lancet body 443 together. For example, the endcap 472 may have a plurality of keepers such as keeper arms 486 extending distally therefrom for coupling the lancet 442 to the endcap during installation of a new lancet. The keeper arms 486 and the lancet body 443 include a coupling mechanism such as a detent. In the depicted embodiment, for example, the keeper arms 486 include inward retainer tabs 488 that can pass through keeper slots 490 formed in a ridge 492 on the lancet body 443 and that catch on the ridge to hold the lancet to the endcap (see FIGS. 44, 46, and 57). Accordingly, the keeper arms 486 and keeper slots 490 preferably align with and are of the same number as the male key tabs 482 (see FIG. 43), so that outward guide tabs 494 on the keepers 486 can be received in the female key guide slots 484 in the carrier 420 (see FIG. 58). Also, there preferably is a flange 496 in the bore 436 in the carriage 422 of the carrier 420 (see FIGS. 57 and 59). The flange 496 catches and holds the outward guide tabs 494 on the keepers 486 after the endcap is rotated in the direction of arrow “r” in FIG. 58 to the position in FIG. 59.

FIGS. 56-62 show the use of the adjustment mechanism and a method of adjusting a lancing device for various penetration depth settings. FIG. 56 shows the carrier 420, the lancet 442, and the endcap 472 ready for assembly. FIG. 57 show the lancet 442 mounted tip-first to the endcap 472, and held there by the keeper arms 486 retained by the ridge 492 on the lancet body 443. This tip-first assembly provides increased safety over other lancing devices. FIG. 58 shows the endcap 472/lancet 442 unit mounted to the carrier 420, with the male key tabs 482 and the keeper guide tabs 494 received in the key guide slot 484. The endcap 472 is then rotated in the direction “r” from the position of FIG. 58 to the locked position of FIG. 59.

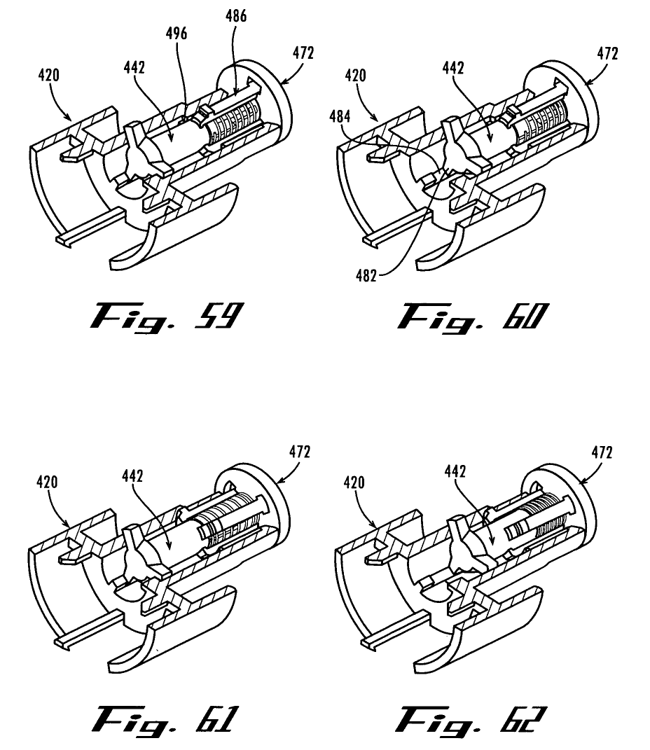

To set the penetration depth, the endcap 472 is rotated to align the desired stop surfaces 478 with the radial arms 474. For example, FIG. 59 shows the endcap 472 adjusted to a deep depth setting, and FIG. 60 shows the lancet 442 in the extended position and stopped at the deep depth (reference the position of the keeper 486). For comparison, FIG. 61 shows the endcap 472 adjusted to a shallow depth, and FIG. 62 shows the lancet 442 in the extended position and stopped at the shallow depth (reference the position of the keeper 486).

Accordingly, it can be seen that the fourth example embodiment provides unique pain-reducing features including the fee-floating of the lancet decoupled from the drive mechanism, the vibratory sensory distraction from stopping the carrier before the lancet, and the adjustable endcap for setting the lancing penetration depth. Preferably, these three features are all included together in a single device. It will be understood, however, that these features may be provided in separate devices independent of each other, or combined with other features described herein or elsewhere, as may be desired in particular applications.

### EXAMPLE 5

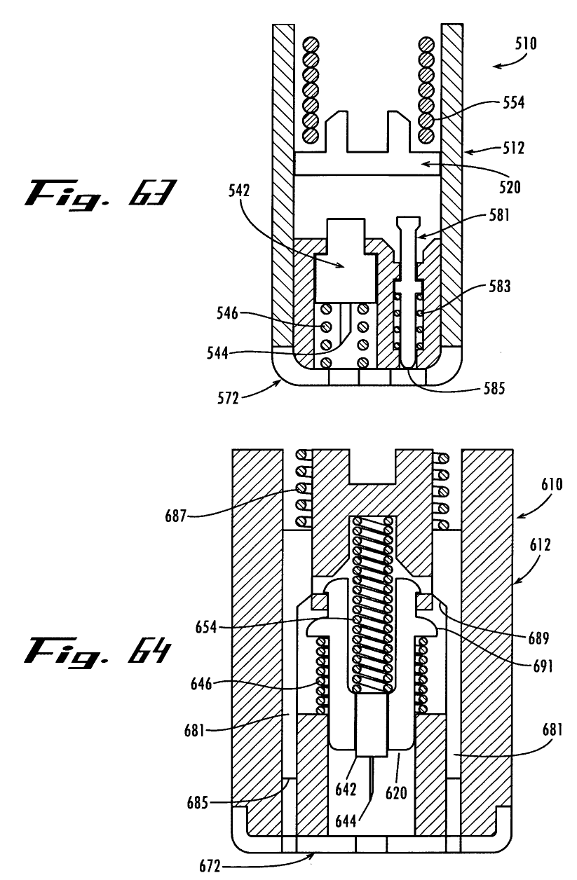

FIG. 63 shows a lancing device 510 according to a fifth example embodiment, the device also capable of generating a sensory distraction but doing so in a different manner. The device 510 includes a housing 512, a drive mechanism including a drive member 520 that is driven by a drive spring 554, a lancet 542 with a tip 544 and a return spring 544, and a stimulator 581 with a tip 585 and a return spring 583. In addition, the device may include a cocking and trigger mechanism (not shown) such as those described herein, and may be provided as a multi-use or disposable lancing device. The stimulator tip 585 may be sharp or blunt, smooth or coarse, single- or multi-pronged, hot or cold, wet or dry, a combination of thereof, or otherwise configured to cause a sensory distraction upon impacting the skin. The lancet 542 and the stimulator 581 are both activated by the single drive member 520. But the stimulator tip 585 contacts the skin just before or simultaneously with the lancet tip 544, causing the desired distraction and thereby reducing the pain perceived from the puncturing by the lancet tip.

The timing can be provided by various means including a “varied lengths” scheme. For example, the stimulator 581 may have a greater length than the lancet 542 and the drive member 520 may have a generally flat contact surface or be otherwise configured for driving the stimulator and the lancet substantially the same distance, as shown. The stimulator tip 585 is preferably nearer than the lancet tip 544 to the endcap 572 at activation, and the stimulator tip and the lancet tip 544 are preferably simultaneously activated by the drive member 520, but this is not necessary. Alternatively, the drive member may have an extension arm, stepped portion, or other lengthened structure for contacting the stimulator before the lancet, regardless of the relative lengths of the stimulator and the lancet.

### EXAMPLE 6

FIG. 64 shows a lancing device 610 according to a sixth example embodiment, the device capable of generating a sensory distraction but doing so in yet another manner. The device 610 includes a housing 612, an endcap 472, a lancet drive mechanism including a drive member 620 (e.g., a lancet holder) that is driven by a drive spring 654, a lancet 642 with a tip 644, and a return spring 646. In addition, the device 620 includes a stimulator 681 with a tip 685 and a return spring 683, and a stimulator drive mechanism including a drive spring 687 that drives the stimulator. The stimulator 681 may be an annular member as shown, or it may be provided in other forms such as those described with reference to the fifth example embodiment. In any case, the lancet 642 and the stimulator 681 are driven so that the stimulator tip 685 contacts the skin just before or simultaneously with the lancet tip 644, causing the desired distraction and thereby reducing the pain perceived from the puncturing by the lancet tip.

The timing can be provided by various means including a “two-stage” scheme in which the lancet 642 and the stimulator 681 are separately driven by dedicated drive mechanisms. The activation of each stage may be manual or automatic. In manual embodiments (not shown), the lancing device has two triggers for manually activating each of the drive mechanisms. For example, the triggers may be positioned generally on opposite sides of the housing for ergonomic use by two fingers (e.g., the thumb and forefinger), with the stimulator trigger having a stiffer spring, longer travel until actuation, etc. than the lancet trigger, so that the stimulator is triggered before the lancet. As another example, the triggers may be positioned adjacent each other for simultaneous actuation (e.g., beside each other or one inside the other), with the stimulator trigger raised relative to the lancet trigger, so that the stimulator is triggered before the lancet.

In automatic embodiments, the device includes a single trigger for activating both drive mechanisms. In one embodiment, the trigger is configured for activating both drive mechanisms at the same time (e.g., using the “varied lengths” scheme, a delay mechanism, etc. to stagger the stimulator and the lancet impacts with the skin). In another embodiment, the trigger is configured to activate the stimulator drive mechanism first and then to activate the lancet drive mechanism at staggered times. And in still another embodiment, the trigger is configured to activate only the stimulator, and then an interlink mechanism activates the lancet drive mechanism. For example, in the depicted embodiment, the lancet drive member 620 has a secondary trigger member such as a finger 691 extending outwardly into the path of an actuator member such as a contact surface 689 defined by the stimulator 681. In operation, the user activates the lancing device main trigger (not shown), which fires the stimulator 681. As the stimulator 681 travels toward the endcap 672, its actuator contact surface 689 comes into engagement with the trigger finger 691 of the lancet drive member 620, releasing the lancet 642 to fire as well. As can be seen in FIG. 64, the stimulator tip 685 contacts the skin just before the lancet tip 644 does.

Then the lancet drive member 620 is retracted by the return spring 646, and the engagement of the trigger finger 691 of the lancet drive member with the actuator contact surface 689 of the stimulator 681 retracts the stimulator as well. Of course, separate dedicated return springs may be used, if desired.

### EXAMPLE 7

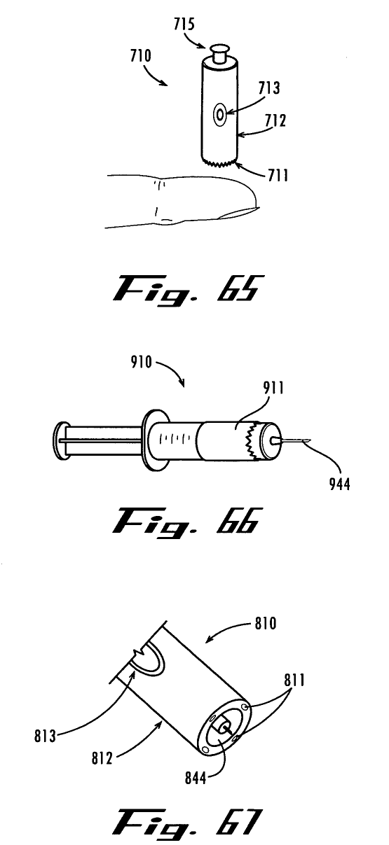

FIG. 65 shows a lancing device 710 according to a seventh example embodiment. The device 710 has a plurality of protrusions such as pointed, blunt, or irregularly shaped teeth 711 at the proximal end of its housing 712 for generating a sensory distraction. Otherwise, the device 710 is similar to conventional lancing devices, and as such includes a cocking mechanism 715 and a trigger mechanism 713. If desired, an adjustable penetration depth endcap mechanism (not shown) may also be provided.

The pointed teeth 711 are preferably arranged in a ring around the edge of the housing 712, but alternatively they may be configured in a pattern or randomly across the proximal end of the housing. In addition, the pointed teeth 711 are preferably integrally formed with the housing 712, but alternatively they may be formed on a face plate or ring that is fastened or adhered to the proximal end of the housing. The number, spacing, sharpness, and length of the teeth 711 may be varied in different embodiments and selected to provide the sensory distraction desired for a particular application or person.

In an alternative embodiment, the pointed teeth are positioned on an axially movable endcap that can be moved in a pumping fashion to engage the skin for generating the sensory distraction. For example, in a manual embodiment the endcap is spring-loaded and biased toward a position adjacent the housing, and includes a handle member for engaging with a finger to manually push the endcap away from the housing in a pumping fashion, thereby compressing the skin once or intermittently, as desired. As another example, in an automatic embodiment the endcap is spring-loaded and biased toward a position adjacent the housing, and the drive mechanism is operably linked to the movable endcap to so that just before or simultaneously with the lancet tip piercing the skin, the endcap contacts the skin at or adjacent the piercing site. And in another alternative embodiment, the teeth are provided on a separate sensory distraction device that can be used repeatedly with a disposable lancing device or with a variety of different lancing devices.

The device 710 may be used in a method of generating a sensory distraction to reduce perceived skin piercing pain. The method includes compressing or pumping the teeth 711 against the skin at or around the lancing site prior to and/or simultaneously with the actuation of the lancing device to puncture the skin. The step of compressing or pumping the teeth 711 against the skin may be done by manually moving the entire device, by manually operating a movable endcap with the teeth, or by automatically operating a movable endcap with the teeth when performing the puncturing. This generates a distracting sensation that reduces the perception of pain resulting from the puncture in many subjects.

### EXAMPLE 8

FIG. 66 shows the proximal end of a lancing device 810 according to an eighth example embodiment. The lancing device 810 includes one or more (four are shown) transducers or contacts 811 for imparting a sensory distraction at and around the lancing site prior to and/or simultaneously with the actuation of the lancing device to puncture the skin. The transducers or contacts 811 are preferably positioned adjacent where the lancet tip 844 extends for puncturing, for example, at the proximal end of the housing 812. Otherwise, the device 810 is similar to conventional lancing devices, and as such includes a cocking mechanism (not shown) and a trigger mechanism 813. If desired, an adjustable penetration depth endcap mechanism (not shown) may also be provided.

The transducers or contacts 811 may be provided by conventional components that are commercially available for generating a sensory distraction that reduces the perception of pain resulting from the puncture in many subjects. For example, the transducers or contacts 811 may be provided by one or more electrodes connected to a battery or piezoelectric generator for imparting a mild to moderate electrical shock or stimulation to the lancing site; one or more acoustical transmitters for generating an acoustical signal; one or more vibrational contacts for transmitting vibration to the skin at the lancing site; one or more heating elements for transmitting heat to the skin at the lancing site; one or more heat exchangers or coils for transferring heat from skin to the heat exchanger and then to a cooling system to cool the skin at the lancing site; one or more flashing and/or colored lights, an object with a mechanism for generating movement or changed visual perception of the object, etc. for generating a visual distraction; and/or a combination thereof. In alternative embodiments, the transducers or contacts 811 are provided by an odoriferous element such as a breakable vial and hammer or other mechanism for releasing a sulfuric or other offensive or pleasing chemical odor; a strong-tasting element that is offensive, pleasing, and/or textured, for example a citrus or garlic flavor in dried, gel, or granular form; and/or a combination of these components to produce a frightening effect such as a sudden change in sensory perception of light, sound, etc. to generate an emotional shock. In these embodiments, the transducers or contacts 811 are positioned on the distal end of the housing, on another part of the housing, or on a separate device from the skin-piercing device.

A power source may be included in the form of replaceable/rechargeable batteries received in the housing, and/or the lancing device may include an AC/DC converter connected to a jack for a power cord for use with house voltage. The number, spacing, position, and type of the transducers or contacts 811 may be varied in different embodiments and selected to provide the sensory distraction desired for a particular application or person. In an alternative embodiment, the transducers or contacts are provided on a separate sensory distraction device that can be used repeatedly with a disposable lancing device or with a variety of different lancing devices.

The device 810 may be used in a method of generating a sensory distraction to reduce perceived skin piercing pain. The method includes applying to the skin at or around the lancing site, a sensory distraction such as a mild-to-moderate electrical shock, a vibration, a hot or cold sensation, an acoustical signal, a visible effect, an odor, a strong taste, an emotional shock, and/or a combination thereof. The method may also include subjecting the patient to additional sensory distractions remote from the puncture site. This generates a distracting sensation that reduces the perception of pain resulting from the puncture in many subjects.

### EXAMPLE 9

FIG. 67 shows a hypodermic syringe 910 according to a ninth example embodiment of the invention. The syringe 910 is similar to conventional syringes except this one includes a spring-driven member 917 that is triggered to impact with the subject's skin at or around the site of penetration of the skin by the needle 944 prior to and/or simultaneously with the puncturing of the skin. The member 917 includes teeth 911 or other sensory distracting elements as described herein with respect to the other example embodiments. In the depicted embodiment, the member 917 is provided by a sleeve that fits over the syringe body, and that has a helical spring (not shown) between the sleeve and the syringe for biasing the sleeve in the neutral position shown. In an alternative embodiment, the syringe 910 includes a interlinking trigger for automatically activating the spring-driven member 917 when the syringe plunger is first pushed.

The syringe device 910 may be used in a method of reducing perceived skin piercing pain. The method includes applying to the skin at or around the lancing site a sensory distraction, for example, by pulling back the member 917 away from the skin and then releasing it so that it is launched into contact with the skin, and operating the syringe to pierce then skin. Impact of the member 911 against the skin generates a sensory distraction that reduces the perception of pain resulting from the puncture in many subjects.

It should be understood that the foregoing relates only to example embodiments of the present invention, and that numerous changes, additions, modifications, and deletions may be made from the example embodiments described without departing from the spirit and scope of the invention as defined by the following claims. For example, the different types of sensory distraction described above can be imparted in connection with any skin puncturing device or procedure, including for example lancing for sampling body fluids, injections, drawing blood, collecting blood and/or plasma from donors, insertion of scopes or other instruments in minimally-invasive surgery and the like, ect.; using a lancet, a syringe, a needle, a cannula; etc. Also, the invention includes generation of a sensory distraction using a separate device from the device used to penetrate the skin, as well as generation of the sensory distraction using the same device used to penetrate the skin. The sensory distraction can comprise vibration, sound, impact, electrical stimulation, light, heat or other signals or effects that are capable of distracting the subject. For example a musical signal may be generated, optionally also incorporating a light signal that may be sequenced to flash in time with the music, to produce a multi-sense sensory distraction.

What is claimed is:

1. A method of reducing perceived pain resulting from puncturing of skin at a puncture site, said method comprising:
generating a sensory distraction at or adjacent the puncture site; and
puncturing the skin at the puncture site simultaneously with or after the generation of the sensory distraction.

2. The method of claim 1, wherein the step of generating a sensory distraction and the step of puncturing the skin are performed using a single device.

3. The method of claim 1, wherein the step of generating a sensory distraction comprises impacting a first element with a second element to generate a vibration for transmission to the puncture site.

4. The method of claim 3, wherein the step of impacting a first element with a second element comprises impacting a stop member with a lancet drive member.

5. The method of claim 1, wherein the step of generating a sensory distraction comprises impacting the puncture site with a stimulator member.

6. The method of claim 5, wherein the step of impacting the puncture site with a stimulator member comprises implementing a varied length scheme for timing the puncturing simultaneously with or after the stimulator impact.

7. The method of claim 5, wherein the step of impacting the puncture site with a stimulator member comprises implementing a two-stage scheme for timing the puncturing simultaneously with or after the stimulator impact.

8. The method of claim 1, wherein the sensory distraction comprises vibration, sound, impact, electrical stimulation, or heat.

9. The method of claim 8, further comprising the step of generating a second sensory distraction remote from the puncture site, wherein the second sensory distraction comprises light, taste, sound, or smell.

10. The method of claim 8, wherein the step of generating a sensory distraction comprises manually pumping or compressing pointed teeth against the skin at the puncture site.

11. A device for penetrating the skin of a human or animal subject, comprising:
means for puncturing the skin at a puncture site; and
means for generating a sensory distraction at or adjacent the puncture site before or simultaneously with the puncturing of the skin.

12. The device of claim 10, wherein the means for puncturing the skin comprises a lancet or a hypodermic needle.

13. The device of claim 10, wherein the means for generating a sensory distraction comprises a first element for impacting a second element and generating a vibration for transmission to the puncture site.

14. The device of claim 13, wherein the first element comprises a stop member and the second element comprises a lancet drive member.

15. The device of claim 13, wherein the device further comprises a housing including the stop member, at least a portion of the housing including a vibration-transmitting material.

16. The device of claim 10, wherein the means for generating a sensory distraction comprises a stimulator member and the means for puncturing the skin comprises a lancet.

17. The device of claim 16, wherein the stimulator member is longer than the lancet, and further comprising a drive spring fog driving both the stimulator member and the lancet.

18. The device of claim 16, further comprising a stimulator drive spring for driving the stimulator member and a lancet drive spring for driving the lancet.

19. The device of claim 18, further comprising a lancet drive member that is driven by the lancet drive spring and that has a trigger finger, wherein the stimulator member includes an actuator contact surface that engages the trigger finger to fire the lancet.

20. The device of claim 18, further comprising a transducer or contact for generating the sensory distraction.

21. The device of claim 18, further comprising a plurality of protrusions for generating the sensory distraction.

22. A lancing device comprising:
a housing;
a lancet defining at least one contact surface;
a drive mechanism including a drive member that engages and drives the lancet; and
an endcap that rotates relative to the housing and that has a plurality of stop surfaces that are selectively aligned with and engaged by the lancet contact surface to limit forward lancet movement.

23. The lancing device of claim 22, wherein the endcap comprises an inner cap and an outer cap that rotates relative to the inner cap.

24. The lancing device of claim 22, wherein the drive member comprises a carriage that receives the lancet and that has a flared proximal section defining a flared bore that receives the endcap stop surfaces not aligned with and engaged by the lancet body engagement surface.

25. The lancing device of claim 22, wherein the endcap has a plurality of keeper arms extending distally there form for coupling the lancet to the endcap, the keeper arms including in ward retainer tabs that can pass through keeper slots formed in a ridge on the lancet and that catch on the ridge to hold the lancet to the endcap, the keeper arms further having outward guide tabs that are received in female key guide slots in the carrier.
